(19) United States
(12) Patent Application Publication
(10) Pub. No.: US 2012/0165626 A1
(43) Pub. Date: Jun. 28, 2012

(54) DEVICES, METHODS, AND KITS FOR DETERMINING ANALYTE CONCENTRATIONS

(76) Inventors: Finkelshtein V. Irina, San Jose, CA (US); James W. Moyer, San Francisco, CA (US); Russell O. Potts, San Francisco, CA (US); Burton H. Sage, San Francisco, CA (US); Robert J. Shartie, San Francisco, CA (US); Donald R. Wilson, San Francisco, CA (US); Bruce D. Wong, San Francisco, CA (US)

(21) Appl. No.: 13/383,809

(22) PCT Filed: Jul. 1, 2010
(86) PCT No.: PCT/US10/40845

§ 371 Date: Jan. 12, 2012

(30) Foreign Application Priority Data
Jul. 13, 2009 (US) 61225153

(51) Int. Cl. A61B 5/1455; A61B 5/145

(52) U.S. Cl. 600/316; 600/309; 600/310; 600/362; 600/365

(57) ABSTRACT

Devices, methods, and kits for measuring or otherwise evaluating the concentration of one or more analytes in a body fluid are described. The devices, methods, and/or kits may be non-invasive. In some variations, a method for measuring the concentration of an analyte in sweat of a subject may comprise contacting a colorimetric membrane with a skin surface of the subject so that the membrane collects a volume of sweat from the skin surface, and analyzing the colorimetric membrane to determine the concentration of the analyte in the collected volume of sweat.

### FIELD

The present application relates generally to measuring or otherwise evaluating (e.g., estimating) the concentration of one or more analytes in a fluid sample. More specifically, the present application relates to devices, methods, and kits that may be used to collect sweat from a skin surface, and to measure the concentration of one or more analytes, such as glucose, in the collected sweat.

### BACKGROUND

Many people around the world suffer from diabetes, and the number of affected people continues to increase. Diabetes is a leading cause of death and can result in broad complications, such as blindness, kidney disease, nerve disease, heart disease, amputation, or stroke.

Diabetes results from the inability of the body to produce or properly use insulin. In simple terms, insulin is a hormone that regulates the level of glucose in the blood and allows glucose to enter cells. In diabetics, glucose cannot enter the cells, so glucose builds up in the blood to toxic levels. Although the cause of diabetes is not completely understood, it is believed that genetics, environmental factors, and viral causes contribute to the incidence of diabetes in the world population.

There are two major types of diabetes: Type 1 and Type 2. Type 1 diabetes (also known as juvenile diabetes) is caused by an autoimmune process destroying the beta cells that secrete insulin in the pancreas. Type 1 diabetes most often occurs in young adults and children. People with Type 1 diabetes are typically required to self-administer insulin using, for example, a syringe or a pen with a needle and cartridge. Continuous subcutaneous insulin infusion via external or implanted pumps is also available. Type 2 diabetes, which is more common than Type 1 diabetes, is a metabolic disorder resulting from the body's inability to make enough insulin or to properly use insulin. People with Type 2 diabetes are typically treated with changes in diet and exercise, as well as with oral medications. Many Type 2 diabetics become insulin-dependent at later stages of the disease. Diabetics using insulin to help regulate their blood sugar levels are at an increased risk for medically-dangerous episodes of low blood sugar due to errors in insulin administration, and/or unanticipated changes in insulin absorption.

It is highly recommended by medical professionals that insulin-using patients practice self-monitoring of blood glucose (“SMBG”). Based upon the level of glucose in the blood, individuals may make insulin dosage adjustments before injection. Adjustments are generally necessary since blood glucose levels vary from day to day for a variety of reasons, such as exercise, stress, rates of food absorption, types of food, hormonal changes (pregnancy, puberty, etc.), and the like. Despite the importance of SMBG, several studies have found that the proportion of individuals who self-monitor at least once a day significantly declines with age. This decrease is likely the result of the most widely used method of SMBG involving obtaining blood from a capillary fingerstick, which can be painful, as discussed below.

The vast majority of equipment used to self-monitor blood glucose is invasive, requiring fingersticks (or lancing alternative sites, such as the forearm) and application of whole blood samples to test strips. Lancing the fingers can be particularly painful over time, and can therefore prevent many users from measuring their blood glucose as frequently as they should. Although non-invasive systems have been developed, some of them exhibit poor correlation to invasive blood glucose measurements, and/or high cost.

In view of the above, it would be desirable to provide additional devices, methods, and kits for measuring or otherwise evaluating the concentration of glucose, and/or other analytes, in a body fluid. It would also be desirable for such devices, methods, and kits to be non-invasive and easy to use. It would further be desirable to provide methods for measuring or otherwise evaluating the concentration of one or more analytes in a body fluid in a relatively short period of time.

### SUMMARY

Described here are devices, methods, and kits for measuring or otherwise evaluating (e.g., estimating) the concentration of one or more analytes in a body fluid. The devices, methods, and/or kits may be non-invasive, and thus may not require painful blood draws (e.g., fingersticks), or their resulting wounds. Moreover, the devices, methods, and/or kits may be used to measure the concentration of one or more analytes in a body fluid relatively efficiently (e.g., in a relatively short period of time).

While the devices, methods, and kits may be configured, as appropriate, to measure or otherwise evaluate the concentration of any analyte or analytes (e.g., glucose, proteins, enzymes, cholesterol, phenylalanine, ketones, etc.) in any body fluid sample (e.g., sweat, blood, serum, urine, saliva, amniotic fluid, etc.), for illustrative purposes, they will be described here with reference to measuring the concentration of glucose in sweat. It should be understood, however, that descriptions provided here with respect to evaluating sweat glucose concentration may also be applied to other suitable analytes and/or body fluid samples. For example, devices, methods, and/or kits described here may be used to test whole blood samples (e.g., relatively small volume samples) for the presence of one or more analytes (e.g., glucose).

Additionally, if so desired, the concentration of an analyte in one body fluid may be used to estimate the concentration of the analyte in another body fluid. For example, a sweat glucose concentration value may be used to estimate a blood glucose concentration value. As an example, a sweat glucose concentration measurement may be correlated to a blood glucose concentration value using one or more algorithms. Thus, a user may be able to determine critical blood glucose values, without having to endure the pain and difficulty that may be associated with obtaining a whole blood sample. Because users may not have to endure any pain associated with testing, it is expected that users will test more frequently than they might with other, more invasive, testing methods. This, in turn, may lead to better compliance with prescribed regimens and, therefore, better clinical outcomes. Moreover, in some cases, the devices described here may be manufactured relatively inexpensively (e.g., by using low-cost materials and/or methods). Accordingly, a user may pay a relatively low cost per test, thereby allowing for more frequent sweat and blood glucose concentration evaluation.

The devices described here typically include one or more membranes. In some variations, the devices may include one or more colorimetric membranes, such that a chemical reaction may occur between an analyte in the collected sweat and one or more chemicals contained in the colorimetric membrane to thereby produce an optically detectable reactant. While devices, methods, and kits are generally described here with respect to colorimetric membranes, it should be understood that devices, methods, and/or kits described here may alternatively or additionally comprise one or more other types of collection and/or analysis supports, such as one or more electrochemical chambers, as appropriate.

In some variations, a colorimetric membrane may be placed into contact with a skin surface and used to collect sweat from the skin surface (e.g., via capillary action or by diffusion or other fluid sequestering means). The concentration of glucose in the collected sweat may then be evaluated (e.g., by imaging the colorimetric membrane after it has collected and reacted with sweat). In certain variations, the devices described here may additionally comprise one or more wicking or collection portions (e.g., layers). The wicking or collection portions may, for example, be located between the colorimetric membrane and the skin surface during use, and may help to wick or collect sweat into the membrane.

In some variations, the devices described here may be in the form of a testing substrate, such as a test strip. While features and characteristics of test strips are described herein, it should be understood that these features and characteristics may also be applied to other types of testing substrates, as appropriate. Testing substrates may have any suitable configuration, including but not limited to circular, oval, square, and rectangular shapes, irregular shapes, uniform thicknesses, and non-uniform thicknesses. In some variations, a testing substrate may be in the form of a tape that may be stored and administered in a roll. The configuration of a testing substrate may depend, for example, on the particular analyte and/or fluid sample being evaluated, the anatomical characteristics of the site that contacts the testing substrate during use, and the methods (e.g., colorimetric or electrochemical) for determining the concentration of the analyte. Moreover, testing substrates may comprise any variety of different suitable materials.

In certain variations, the devices, methods, and/or kits described here may be used to collect a volume of sweat that is relatively small. For example, the volume of sweat may be less than about 10 microliters (e.g., about 5 microliters, about 3 microliters, about 1 microliter, about 0.8 microliter, about 0.5 microliter, about 0.3 microliter, about 0.1 microliter, or less). In some cases, the volume of the sweat may be less than about 1 nanoliter. The concentration of glucose in the sweat may be, for example, from about 0.1 mg/dL to about 10 mg/dL (e.g., from about 0.1 mg/dL to about 5 mg/dL). Glucose concentration may be measured at these levels or in certain variations, may be measured at levels of, for example, less than about 0.5 mg/dL.

Some variations of methods for measuring the concentration of an analyte in sweat of a subject may comprise placing a membrane (e.g., a colorimetric membrane or electrochemical strip) into contact with a skin surface of the subject so that the membrane or strip collects a volume of sweat from the skin surface, and analyzing the membrane or strip to determine the concentration of the analyte in the collected volume of sweat.

The membrane may be analyzed using any of a number of different methods. As an example, an optical system may be used to evaluate spectral emissions (e.g., when fluorescence is used), or the spectral absorption or reflection, of a colorimetric membrane. As another example, light from one or more light-emitting diodes may be applied to a colorimetric membrane, and/or one or more photodiodes may be used to detect light reflected from a colorimetric membrane. As an additional example, an optical system may be used to evaluate the intensity of spectral light reflected from a colorimetric membrane. As another example, an optical system may be used to evaluate the intensity of monochromatic light reflected from a colorimetric membrane. In certain variations, a densitometer may be used to analyze a colorimetric membrane. In some variations, light from a laser, and/or a wide spectrum light source, may be directed to a colorimetric membrane. In certain variations, a charge-coupled device (CCD), a CMOS-based detector, and/or a camera may be used to image a colorimetric membrane. Some methods may include scanning a colorimetric membrane to determine the optical density of at least one colored portion of the membrane. In certain variations, the optical transmission property of a colorimetric membrane may be evaluated.

In some variations, a colorimetric membrane may include one or more spots generated by a chemical reaction between the analyte and chemicals contained in the colorimetric membrane, where the chemical reaction occurs when the colorimetric membrane contacts the skin surface. The method may comprise discriminating the background color of the membrane from the spot(s). This may, for example, help to distinguish the target analyte(s) from contaminants. Alternatively or additionally, the appearance of spots on the colorimetric membrane may be used to estimate the sweat rate of the subject.

Contacting the membrane with the skin surface may comprise holding the membrane against the skin surface. The membrane may, for example, be in contact with the skin surface for at most about one hour (e.g., at most about 30 minutes, at most about 10 minutes, at most about 5 minutes, at most about 4 minutes, at most about 3 minutes, at most about 2 minutes, at most about 1 minute, at most about 30 seconds, at most about 20 seconds, at most about 10 seconds, at most about 5 seconds). Alternatively or additionally, the membrane may, for example, be in contact with the skin surface for at least about 1 second (e.g., at least about 5 seconds, at least about 10 seconds, at least about 20 seconds, at least about 30 seconds, at least about 1 minute, at least about 5 minutes, at least about 10 minutes, at least about 30 minutes).

In some variations, the collected volume of sweat may saturate the membrane. In certain variations, the collected volume of sweat may be collected by a portion of the membrane, and the method may comprise analyzing the portion of the membrane.

In some variations, the analyte may comprise glucose. The method may further comprise calculating or estimating the concentration of glucose in blood of the subject (e.g., using at least one algorithm that converts the concentration of glucose in sweat to the concentration of glucose in blood). In certain variations, a colorimetric membrane may comprise a first component (e.g., glucose oxidase) that converts glucose to hydrogen peroxide. The colorimetric membrane may further comprise a second component (e.g., a peroxidase, such as horseradish peroxidase) that reacts with the hydrogen peroxide. The colorimetric membrane may also comprise a third component comprising an indicator that changes color in the presence of hydrogen peroxide. The indicator may, for example, comprise an oxidizable dye or a dye couple, such as meta [3-methyl-2-benzothiazolinone]N-sulfonyl benzenesulfonate monosodium combined with 8-anilino-1-naphthalene sulfonic acid ammonium.

The method may further comprise inducing sweat prior to collecting the volume of sweat from the skin surface. Sweat may be induced, for example, by administering pilocarpine to the skin surface.

### BRIEF DESCRIPTION OF THE DRAWINGS

FIG. 1A is a top view of a variation of a test strip; and FIG. 1B is a bottom view of the test strip of FIG. 1A.

FIG. 2A is a perspective view of a variation of a test region of a test strip, and FIG. 2B is a cross-sectional view of the test region of FIG. 2A, taken along line 2B-2B.

FIGS. 2C-2E are cross-sectional views of additional variations of test regions of test strips.

FIGS. 3A-3D are perspective views of different variations of spreading layers of test strips.

FIG. 4 is a flowchart representation of a variation of a method for making a test strip.

FIGS. 5A-5C are different views of a variation of a test well array that may be used to determine the concentration of glucose in a single sweat bolus.

FIG. 6A is cross-sectional view of a portion of a test well array. FIG. 6B is a flowchart representation of a variation of a method for making a test well array.

FIG. 7 is an illustrative top view of a variation of a meter for measuring the concentration of an analyte in a fluid sample.

FIG. 8 is a flowchart representation of a variation of a method for evaluating the concentration of glucose in blood of a subject.

FIG. 9A is a photograph of a colorimetric membrane contacting a finger of a subject, and

FIG. 9B is a photograph of the colorimetric membrane of FIG. 9A after its color has changed as a result of contact with the finger.

FIGS. 10A and 10B are photographs of colorimetric membranes after different exposure times to a skin surface.

FIGS. 10C-10H are photographs of colorimetric membranes after different exposure times to a skin surface, with each colorimetric membrane having one side wrapped in Parafilm®.

FIG. 11A is a photograph of a colorimetric membrane after a glucose solution of known concentration has been applied to the colorimetric membrane using an inkjet printer; FIG. 11B is an image of FIG. 11A taken from a red video channel; FIG. 11C is a photograph of the colorimetric membrane of FIG. 11A; and FIG. 11D is a graphical representation of the grey scale intensity of a selection of spots shown in FIG. 11C.

FIG. 11E is a photograph of portions of six test strips that have been exposed to glucose solutions having different concentrations; FIG. 11F depicts the red channel component of FIG. 11E;

FIG. 11G depicts the blue channel component of FIG. 11E; and FIG. 11H depicts the green channel component of FIG. 11E.

FIG. 11I is a graphical representation of the optical intensity of each profile of FIGS. 11F-11H along a horizontal line drawn through each profile vs. distance along the profile.

FIGS. 11J-11O each plot the relationship between the optical signal of a single channel vs. glucose concentration or the base 10 logarithm of glucose concentration.

FIG. 11P is a histogram depicting image data for the red channel component of FIG. 11E;

FIG. 11Q is a histogram depicting image data for the green channel component of FIG. 11E; and FIG. 11R is a histogram depicting image data for the blue channel component of FIG. 11E.

### DETAILED DESCRIPTION

Devices, methods, and kits for sensing and/or measuring glucose in sweat are described. In general, sweat may be collected from a skin surface of a subject (e.g., a patient) using, for example, a testing substrate such as a test strip. The collected sweat may then be evaluated to determine its concentration of glucose. In some cases, the test strip may be a colorimetric test strip. For example, the test strip may comprise one or more colorimetric membranes. The membrane or membranes may contain one or more reagents that change color as a function of the concentration of glucose in the collected sweat. After sweat has been collected for a certain period of time (which may be relatively short), the color of the membrane may be measured (e.g., using optical techniques, as discussed further below). If so desired, the resulting measurement may then be correlated to a blood glucose concentration. The devices, methods, and kits will now be described below. While certain components and materials will be described, it should be understood that other appropriate components and materials may alternatively or additionally be used in some variations. For example, in certain variations, one or more components and/or materials described in U.S. patent application Ser. Nos. 11/159,587 (published as US 2006/0004271 A1) and/or 11/451,738 (published as US 2007/0027383 A1) may be used. Both of these references are incorporated herein by reference in their entirety.

### Devices

### A. Test Strips

Any suitable test strip or other testing substrate may be used to measure the concentration of glucose in sweat. It should be noted again that while the example of measuring the concentration of glucose in sweat and then correlating the sweat concentration to a blood concentration is discussed in detail here, the devices, methods, and kits described here may be used to measure or otherwise evaluate the concentration of any analyte in any fluid sample, as appropriate.

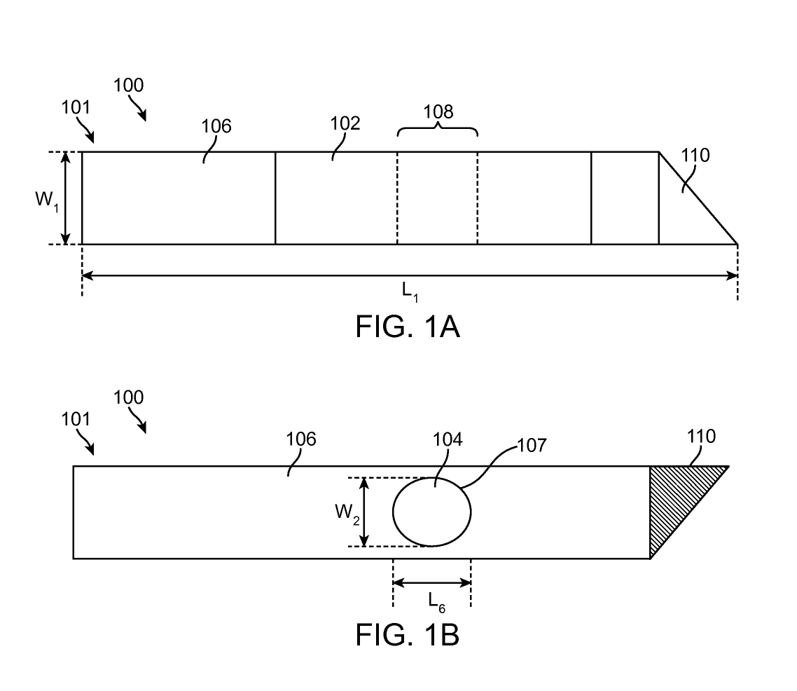

FIGS. 1A and 1B show one variation of a test strip (100). FIG. 1A shows the top surface (101) of test strip (100), and FIG. 1B shows the bottom surface (103) of test strip (100). As shown in FIGS. 1A and 1B, test strip (100) comprises a membrane (104) generally located in a test region (108), and a base (106). Upon contacting test strip (100), a fluid sample may flow into membrane (104), where one or more reagents may be used to detect a characteristic (e.g., presence, concentration, absolute quantity, reactivity, etc.) of a target analyte. In some variations, a detection system or other appropriate device or method may then be used to interrogate the test strip (e.g., optically, chemically, and/or electrically) and convey information about the analyte to the user.

In certain variations, membrane (104) of test strip (100) may be a colorimetric membrane, such that the above-described measured property of the target analyte may be conveyed via color changes of the membrane. In some variations, a colorimetric membrane may comprise a substrate or matrix material and one or more reagents selected to react with or in the presence of one or more analytes. When a fluid sample comprising one or more of the specific analytes is applied to the colorimetric membrane, the color of the colorimetric membrane may change, thereby providing a visual indication of the presence of the analyte or analytes in the fluid sample. In some cases, the color change (e.g., the change in the optical absorption and/or reflection spectrum) may then be evaluated and/or measured (e.g., to determine the concentration of the analyte or analytes in the fluid sample). Examples of measurement devices that may be used to measure and/or evaluate such a change, as well as examples of colorimetric membranes, are described in further detail below.

Test strip (100) may also comprise a spreading layer. In some variations, the spreading layer may extend across a substantial portion of test strip (100), such as at least about 20% of the length of test strip (100). In certain variations, a spreading layer may extend over the entirety of a membrane (e.g., membrane (104)). In other variations, a spreading layer may only extend over one or more portions of a membrane. In variations in which a test strip comprises a membrane and a spreading layer, the membrane may be located anywhere along the length of the spreading layer. For example, the membrane may be generally centered relative to the spreading layer. The spreading layer may be used to help distribute a fluid sample on the test strip, so that the sample does not over-saturate a single location of a membrane of the test strip. Spreading layers are described in additional detail below.

Membrane (104) (and, e.g., a spreading layer) may be mounted on base (106). Base (106) may provide additional structural support and ease of handling. However, other variations of test strips may have different configurations that may or may not include a base. For example, in certain variations, instead of including a base, a test strip may comprise a spreading layer and a membrane in the form of a tape that is enclosed within a cartridge as a spool, and installed in a device requiring little or no manual handling.

Referring again to FIGS. 1A and 1B, base (106) includes a window (107) that is located within test region (108). In some variations, window (107) may expose membrane (104) for application of sample to membrane (104) for analysis (e.g., by optical, chemical, or electrical means). Window (107) may have any suitable shape or size. In some variations, window (107) may be molded at the same time that base (106) is formed, while in other variations window (107) may be cut out after base (106) is formed.

As shown in FIG. 1A, test strip (100) has a length L₁ and a width W₁. As described previously, a spreading layer may be situated at any appropriate location along a test strip. For example, a spreading layer may be located along the length of base (106). In some variations, length L₁ may be from about 1 centimeter to about 8 centimeters, and/or width W₁ may be from about 0.3 centimeter to about 4 centimeters.

Referring again to FIGS. 1A and 1B, test region (108) is located within membrane (104). Additionally, window (107) has a longitudinal dimension L₆ (e.g., length or diameter, depending on the shape), and a width W₂, where L₆ and W₂ may be, for example, from about 0.1 centimeter to about 3 centimeters.

Test strips may comprise any appropriate number of layers. For example, a test strip may comprise the same number of layers as test strip (100), or may comprise fewer layers or more layers. Different exemplary variations of test strips comprising different layers, configurations, and compositions are described in further detail below.

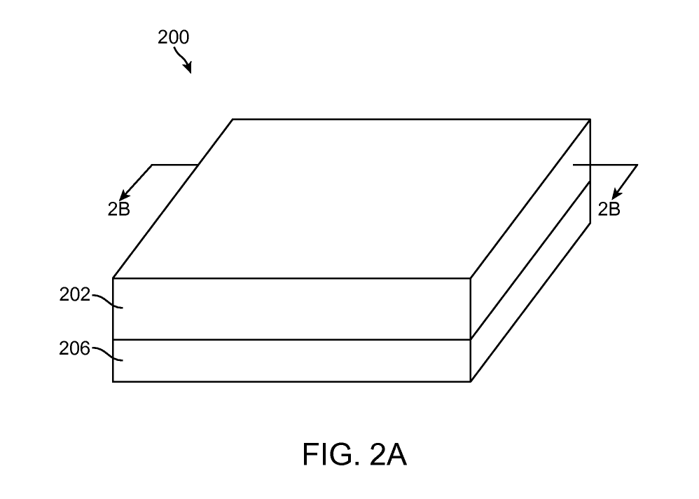

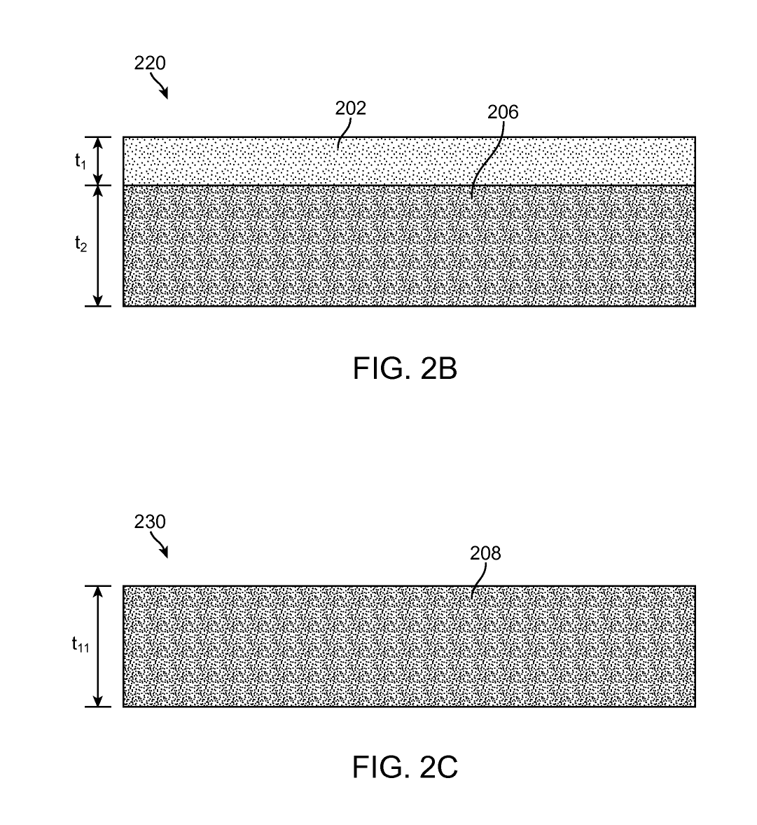

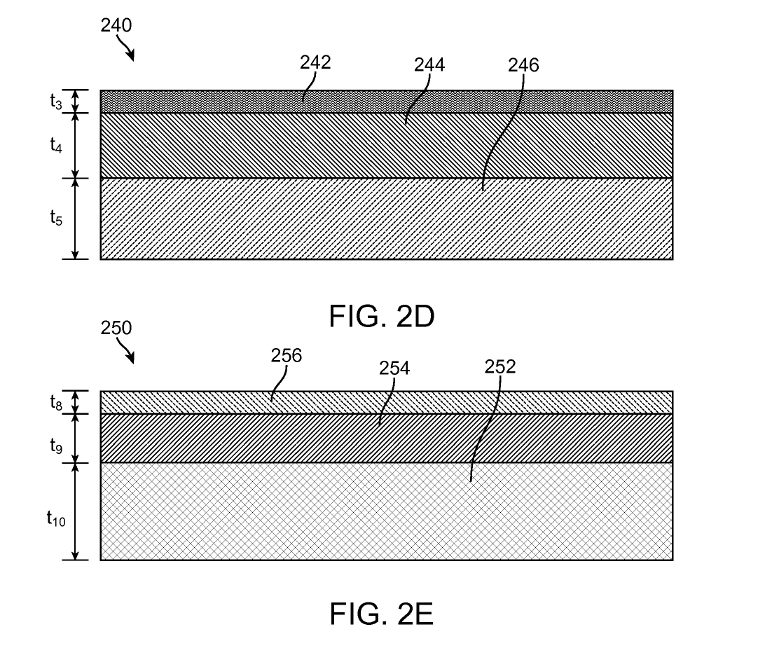

A variation of a test region (200) of a test strip is depicted in FIGS. 2A and 2B. As shown there, test region (200) comprises a spreading layer (202) and a membrane (206). During use, a fluid sample, such as blood or sweat, may come into contact with spreading layer (202), such that the fluid sample may be distributed laterally as it flows to membrane (206). The target analyte may then be detected in membrane (206).

In some variations, a test strip may comprise one or more layers that separate a fluid sample source (e.g., a source of blood, or a skin surface) from a membrane of the test strip. For example, some variations of test strips may have two separating layers, such as a spreading layer and a porous layer (e.g., test region (240) of the test strip depicted in FIG. 2D), or may have just one separating layer, such as a porous spreading layer (e.g., test region (220) of the test strip depicted in FIG. 2B).

The layers of a test strip may have the same thickness, or varying thicknesses throughout. For example, the test strip test region (220) shown in FIG. 2B has two layers of different thicknesses. As shown there, test region (220) comprises a spreading layer (202) having a thickness t₁ which may, for example, be from about 5 microns to about 700 microns (where one “micron” is equivalent to one micrometer). Additionally, test region (220) comprises a membrane (206) having a thickness t₂, where t₂ may be, for example, from about 5 microns to about 500 microns. It should also be noted that some variations of test strips may comprise multiple layers of different areas. For example, a test strip may comprise a middle layer with a smaller area located between two layers (e.g., a top and bottom layer) each having a larger area.

FIG. 2C depicts a test region (230) of a test strip comprising just one porous layer (208). In such cases, the layer may have a single function, or may have multiple functions. For example, in some variations, the layer may function as a membrane (e.g., a colorimetric membrane). In some such variations, only the reagent that is in close proximity to the placement of the fluid sample reacts in the presence of, and thereby detects, analyte in the sample. Optionally, the layer may function both as a spreading layer and as a membrane, such that a fluid sample may traverse across the surface prior to contacting and reacting with the reagent. The reagent or reagents may be distributed throughout the porous layer, or may, for example, be located in a sub-region (e.g., a sub-layer) of the porous layer. In other variations, and as described briefly above, one or more layers may separate the fluid sample source (e.g., a skin surface) from the membrane. Porous layer (208) has a thickness t₁₁, where t₁₁ may be, for example, about 5 microns to about 500 microns.

The test region (240) of another variation of a test strip is shown in FIG. 2D. As shown there, test region (240) comprises a spreading layer (242), a porous layer (244), and a membrane (246). Spreading layer (242) has a thickness t₃, where t₃ may be, for example, from about 5 microns to about 700 microns. Additionally, porous layer (244) has a thickness t₄, where t₄ may be, for example, from about 5 microns to about 500 microns, and membrane (246) has a thickness t₅, where t₅ may be, for example, from about 5 microns to about 500 microns.

The thickness of any layer in a test strip, such as one of the test strips described above, may be based on any of a number of factors. For example, the thickness of a layer may depend on the fluid characteristics of the sample to be tested, the porosity of the layer (and/or other layers), the quantity of the fluid sample required to provide an accurate detection, the sensitivity of the membrane to the target analyte, and any characteristics that may impact the fluid flow from the sample source (e.g., a skin surface). As an example, in certain variations, the thickness of the spreading layer may be selected based on the features of the fluid sample being tested, and/or based on the target analyte. In some variations, the spreading layer may have a thickness of about 5 microns to about 700 microns. The material composition of each layer may also be chosen based on optical, electrical, and/or capacitive characteristics, and/or one or more other characteristics.

As described previously, membranes that are used in the devices described here may have any appropriate size and shape (e.g., rectangular, circular, oval, etc.). In some variations, a membrane may have a thickness of about 5 microns to about 400 microns (e.g., about 5 microns to about 30 microns, about 25 microns to about 50 microns, about 50 microns to about 75 microns, about 75 microns to about 100 microns, about 100 microns to about 150 microns, about 150 microns to about 350 microns, about 200 microns to about 300 microns, about 225 microns to about 275 microns). For example, a membrane may have a thickness of about 5 microns, about 10 microns, about 25 microns, about 50 microns, about 75 microns, about 100 microns, about 115 microns, about 125 microns, about 140 microns, about 145 microns, about 150 microns, about 170 microns, about 178 microns, about 200 microns, about 250 microns, about 280 microns, about 305 microns, about 318 microns, about 330 microns, about 343 microns, or about 350 microns. In some cases, the thickness of a membrane may be selected based on the analyte that is being evaluated.

FIG. 2E depicts a test region (250) of another variation of a test strip. As shown there, test region (250) comprises a membrane (256), as well as a wicking layer (254) and a sink layer (252) beneath membrane (256). During use, wicking layer (254) may draw excess fluid sample from membrane (256) to sink layer (252). As shown in FIG. 2E, membrane (256) has a thickness t₈, where t₈ may be, for example, from about 5 microns to about 500 microns. Additionally, wicking layer (254) has thickness t₉. In some variations, t₉ may be from about 5 microns to about 500 microns. Moreover, sink layer (252) has a thickness t₁₀. In certain variations, t₁₀ may be from about 50 microns to about 500 microns.

Wicking layer (254) may be composed of any appropriate absorbent material or materials, such as hydrophilic treated polycarbonate or polyester, or any other material or materials that may provide for relatively efficient fluid transfer from membrane (256) to sink layer (252). For example, wicking layer (254) may be composed of hydrophilic track etched polycarbonate, such as the polycarbonate track etch (PCTE) series of materials from Sterlitech, of Kent, Wash. Alternatively, wicking layer (254) may be composed of one or more hydrophilic monofilament open mesh fabrics, such as the PETEX® series of materials from Sefar Filtration, of Depew, N.Y. In some variations, sink layer (252) may be in the form of a chamber configured to contain excess fluid sample transferred via wicking layer (254). A sink layer (252) that acts as a chamber may be made of, for example, an injection molded thermoplastic, such as polycarbonate, acrylic, acrylonitrile butadiene styrene (ABS), or polystyrene. In some variations, sink layer (252) may comprise one or more porous materials that absorb a greater quantity of fluid than the wicking layer. An absorbent sink layer (252) may be composed of any appropriate highly absorbent material(s), such as Porex Fiber Media or Porex Sintered Porous Media from Porex Corporation of Fairburn, Ga.

Including an additional wicking layer (254) and sink layer (252) may, for example, enhance the precision and accuracy of analyte detection by membrane (256). As an example, the presence of the wicking layer and sink layer may prevent the membrane from becoming over-saturated with the fluid sample and providing an invalid measurement. For example, during use the volume of sweat produced by one sweat gland may over-saturate the reagent(s) in membrane (256). Such over-saturation may lead to an erroneous reading. However, by including a wicking layer (254) and a sink layer (252), excess sweat may be removed from membrane (256), thereby enhancing the accuracy of the sweat glucose concentration measurement. It should be understood, however, that these additional layers below the membrane are optional (e.g., depending on the saturation level of the reagent(s) and the desired detection precision).

The different layers of a test strip may be attached or otherwise coupled to each other in a variety of ways. In some variations, the individual layers may be bonded with one or more adhesives, such as pressure sensitive or heat activated acrylic adhesives, such as the ARcare® series from Adhesives Research of Glen Rock, Pa. The adhesive(s) may be transparent or opaque, as appropriate for the detection technique of the membrane. In some variations, test strips that are optically interrogated may be bonded with a transparent adhesive. In certain variations, the adhesive(s) may be applied throughout the test strip, except in the proximity of the test region. This may prevent any cross-contamination between the adhesive(s) and the sample. Additionally, in the case of methods in which a test strip is optically probed, using an opaque adhesive away from the test region may minimize optical interference. Test strip layers may also be attached to each other by electrostatic forces, welding, clip compression, hook-and-loop fasteners, and any other suitable mechanism that ensures secure and reliable fluid contact between layers.

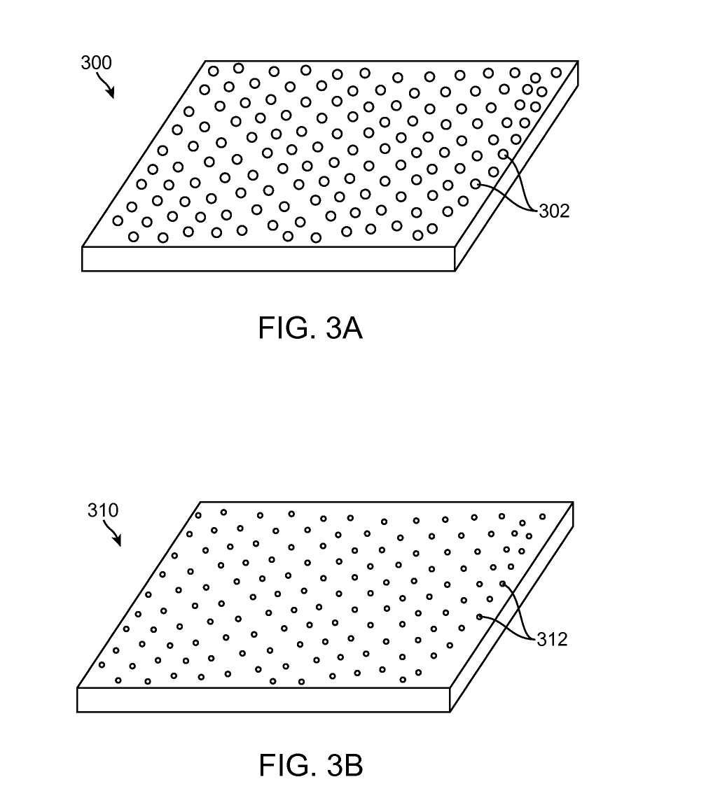

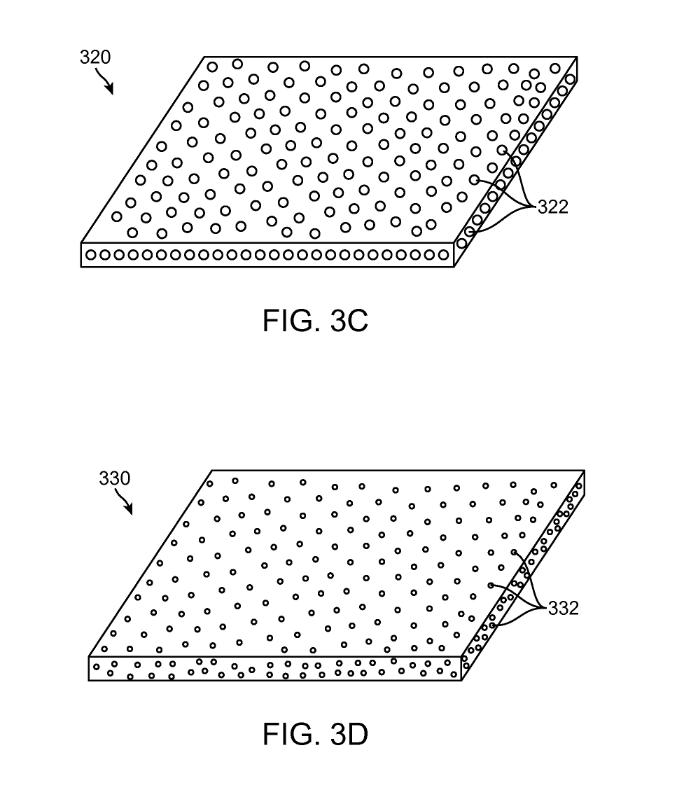

As described above, in some variations of test strips, the fluid sample (here, sweat) initially contacts a spreading layer. Portions of different variations of spreading layers are depicted in FIGS. 3A-3D. The spreading layer may act to wick sweat across the test region, so that the sweat can be evenly distributed across a membrane of the test region. This, in turn, may reduce the saturation of local regions. In such variations, the spreading layer may be selected to have a capillary structure that is strong enough to draw sweat from the skin, but that is weaker than the capillary structure in the layers that lead to the membrane. As a result, sweat may be efficiently drawn from the spreading layer into the membrane.

Some variations of spreading layers may be porous. The pores in a spreading layer may all be of substantially the same size, or at least some of the pores may differ in size. In certain variations, a pore may range in size from about 2 microns to about 350 microns (e.g., about 2 microns to about 20 microns, about 50 microns to about 250 microns, about 50 microns to about 150 microns, about 100 microns to about 150 microns). Alternatively or additionally, the pores in a spreading layer may have a mean size of about 100 microns.

FIG. 3A shows a spreading layer (300) including pores (302) in the form of through-holes extending substantially straight through one side of the spreading layer to the other side. A similar variation is shown in FIG. 3B, in which the through-hole pores (312) are of a smaller diameter than the variation shown in FIG. 3A. Pore size may be selected, for example, based on the fluid characteristics of the target sample or samples, and/or may be tailored to efficiently transport one or more specific types of fluid samples. Through-hole pores may allow for the formation of a direct fluid connection from one side of the spreading layer to the other.

As shown in FIGS. 3C and 3D, in some variations a spreading layer may be similar to a sponge, with pores (322) and (332) extending in all directions throughout the thickness of the spreading layer. Such sponge-like spreading layers may be more absorbent, laterally distributing the fluid sample, and may allow for the formation of an indirect fluid connection from one side of the spreading layer to the other.

A spreading layer may comprise pores that are all of approximately the same size, or may comprise at least some pores having different sizes. Pores may be uniformly distributed throughout a spreading layer, or may be located in one or more specific regions of a spreading layer. In variations of spreading layers including pores of different sizes, the pores may be uniformly distributed, or may be distributed in a gradient, for example, such that the pores are grouped by size.

Depending on, for example, the fluid sample, the spreading layer may comprise any of a variety of different materials or combinations thereof. Examples of materials which may be suitable for use in a spreading layer include hydrophilic woven fabrics (e.g., Tetko mesh #7-280/44, from Sefar America Inc. (formerly Tetko Inc.)), sintered hydrophilic materials (e.g., from Porex Corporation, Fairburn, Ga.), and membranes (e.g., Nuclepore™ track-etched polycarbonate membranes from Whatman/GE Healthcare, such as Nuclepore #113516, 12 micron hydrophilic membrane, or the PCTE series of materials from Sterlitech, of Kent, Wash.). Membrane materials also are described, for example, in U.S. patent application Ser. Nos. 11/159,587 (published as US 2006/0004271 A1) and 11/451,738 (published as US 2007/0027383 A1), both of which were previously incorporated herein by reference in their entirety. In some variations, a spreading layer may comprise one or more heat-sintered plastics (e.g., polyethylene, polypropylene, etc.) that have been rendered hydrophilic by pre- or post-treatment with one or more surfactants. An example of such a material is a porous polyethylene treated with sodium methyl oleoyl taurate and available from Porex Corporation (Fairburn, Ga.). One advantage of this material is that it has relatively strong absorption, which can cause fluid to be drawn away from the surface, where it might otherwise transfer to objects or people it contacts. Other appropriate materials may alternatively or additionally be used.

As described above, some variations of devices described here may comprise one or more membranes. In some cases, a membrane may comprise a colorimetric membrane. For example, the membrane may be used to wick small volumes of sweat from a skin surface, to provide a matrix for one or more reagents that are to come into contact with the collected sweat, and/or to allow for optical measurement of color. Additionally or alternatively, as described above, a spreading layer or porous layer may be used to wick small volumes of sweat from a skin surface and transfer it through capillary action to the membrane.

A colorimetric membrane may comprise any of a variety of different materials. The selected materials may depend on a number of factors, such as the sample volume required for testing, color development, wicking action, optical properties, and desired shelf life. Examples of materials that may be appropriate include charged nylon membranes (e.g., from General Electric Company and Pall Corporation), polysulfone membranes (e.g., HT Tuffryn® Polysulfone Membrane Disc Filters from Pall Corporation), nitrocellulose membranes (e.g., from Sartorius AG), and the like.

In some variations, the material or materials that are used in a colorimetric membrane may be selected based on the reagent(s) that are used to detect the target analyte(s). Alternatively or additionally, the material(s) may be selected based on one or more indicator dyes that may be added to the colorimetric membrane. As an example, a membrane material may be selected based on its ability to retain certain reagent(s) and/or indicator dye(s). In some variations, a reagent may be fixedly cross-linked to the membrane material. For example, in some variations, an enzyme reagent may be immobilized using glutaraldehyde. Alternatively or additionally, a colorimetric membrane may comprise a reagent that is not fixedly cross-linked to the membrane, such that the reagent is mobile. In certain variations, membrane materials, as well as reagents and/or indicator dyes, may be selected based on their non-toxicity and safety for human contact.

As shown above, in some variations, a test strip membrane, and/or any other test strip components, may be porous. Porous membranes may comprise pores of a relatively uniform size, or may comprise pores of different sizes. In certain variations, a porous membrane may include pores having a size of about 0.2 micron to about 5 microns (e.g., about 0.45 micron to about 3 microns, about 0.65 micron to about 1.2 microns, 0.8 micron to about 1.2 microns). For example, a pore may have a size of about 0.2 micron, about 0.45 micron, about 0.65 micron, about 0.8 micron, about 1.2 microns, about 3 microns, or about 5 microns. In some variations, a porous membrane may have at least two different regions having different average pore sizes. For example, one side of a porous membrane may have an average pore size of about 0.2 micron, while an opposite side of the porous membrane may have an average pore size of about 20 microns.

A test strip may comprise one membrane or a combination of membranes, including, for example, any of the membranes described here. Any material having any suitable pore distribution (e.g., a pore distribution that promotes efficient unidirectional fluid flow) may be used in a test strip.

As discussed above, in some variations, a colorimetric membrane may comprise one or more reagents that are selected to react with one or more specific analytes to produce a certain color or colors. For example, in cases in which sweat glucose concentration is being evaluated, a colorimetric membrane may comprise one or more reagents that are selected to provide optimal performance in the range of expected sweat glucose concentrations. A colorimetric membrane may comprise, for example, any suitable combination of enzymes, dyes, and/or additives for detecting a target analyte or analytes.

As an example, some variations of colorimetric membranes for evaluating sweat glucose concentration (and blood glucose concentration therefrom) may comprise one or more reagents that react with glucose to cause a detectable color change. For example, a reagent may comprise a component (e.g., glucose oxidase) that converts glucose to hydrogen peroxide, as well as one or more components that detect the resulting hydrogen peroxide. An example of such a hydrogen peroxide-detecting component is a peroxidase (e.g., horseradish peroxidase) acting in conjunction with an indicator that changes color in the course of the reaction. The indicator may, for example, be an oxidizable dye or a dye couple. In some variations, the indicator may comprise meta [3-methyl-2-benzothiazolinone]N-sulfonyl benzenesulfonate monosodium combined with 8-anilino-1-naphthalene sulfonic acid ammonium (MBTHSB-ANS). The peroxidase may catalyze the oxidation of the indicator in the presence of hydrogen peroxide.

In certain variations in which a specific analyte is being detected, the reagent may be selected for optimal use with certain concentration ranges of that analyte. For example, in the case of glucose, the reagent may be optimized for measurement of sweat glucose concentrations in the range of 0.1 mg/dL to 10 mg/dL (e.g., 0.5 mg/dL to 10 mg/dL, 0.5 mg/dL to 4 mg/dL). Additionally, the shelf life of a reagent may, for example, be from about 6 months to about 2 years.

In certain variations, one or more reagents may be coated onto a colorimetric membrane. This may, for example, result in maximized color development while requiring application of only a minimal sample volume of sweat.

### B. Methods of Making Test Strips

Test strips or other testing substrates may be made using any appropriate method. Typically, a test strip may be designed so that it is easy to use and/or manufacture. In certain variations, a test strip may comprise a colorimetric membrane mounted on a holder. A test strip may be designed both to position a colorimetric membrane close to a skin surface during use, and to register the colorimetric membrane with regard to a reading device (e.g., an optical device) when the color is read.

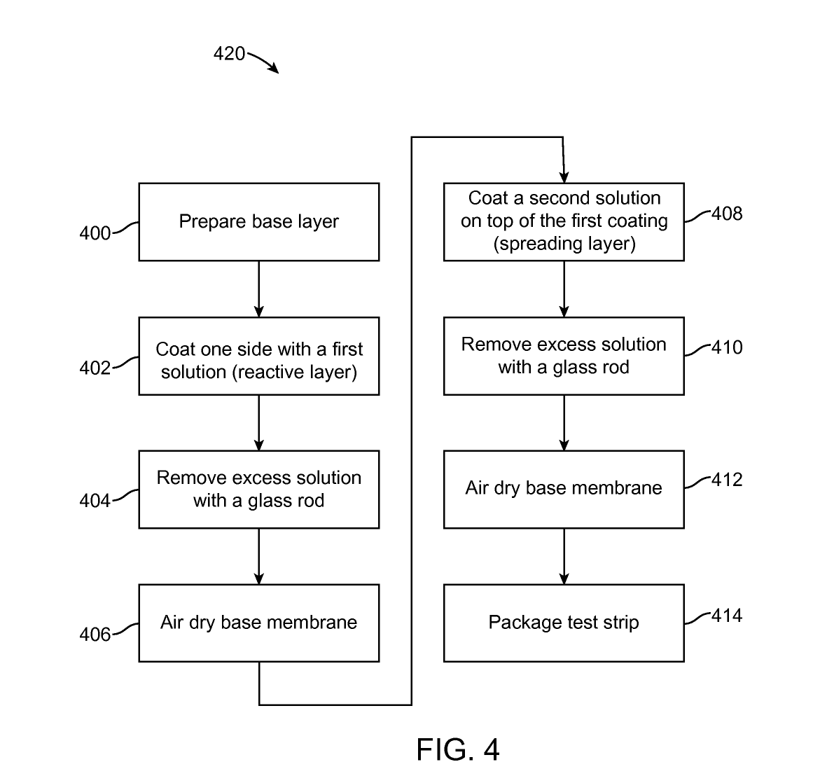

FIG. 4 illustrates one variation of a method (420) that may be used to make test strips, such as the test strips described above. As shown there, method (420) comprises cleaning and preparing a base layer or substrate for subsequent layer deposition (400). Next, the base layer is coated with a first solution on one side, to form a reactive layer (402). Excess solution is then removed (e.g., by washing or physical abrasion, or with a glass rod) (404). The base layer with the deposited reactive layer is dried, such as by air-drying (406). An oven or otherwise elevated desiccating environment may be used to expedite the drying time. Next, a second solution, such as the material for the spreading layer, is applied on top of the first coating (408). Excess solution is again removed (e.g., with a glass rod) (410) and the base membrane is again dried (e.g., by air-drying) (412), as previously described. Additional layers may be applied by repeating the above method. When all desired layers have been applied, the test strip may optionally be packaged (e.g., to preserve cleanliness and for shipping). While FIG. 4 depicts one variation of a method of making a test strip, this method variation is only exemplary, and other appropriate methods may also be used.

### C. Test Well Array

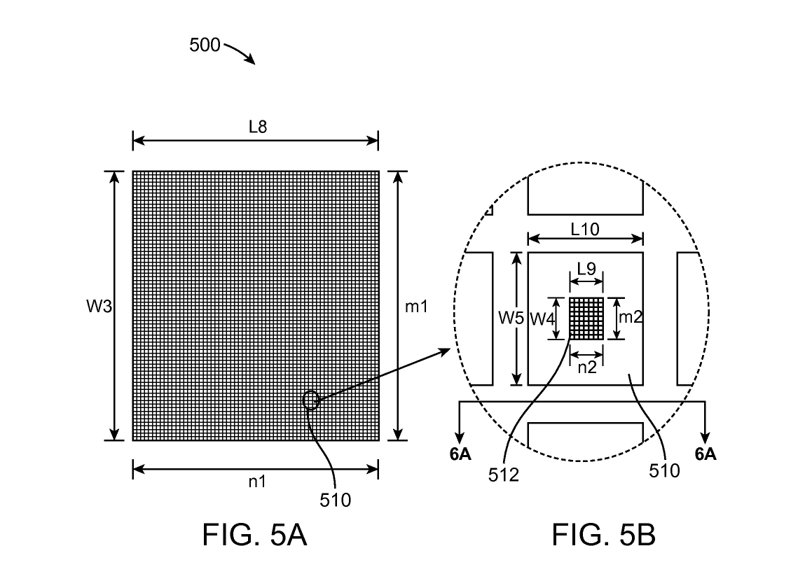

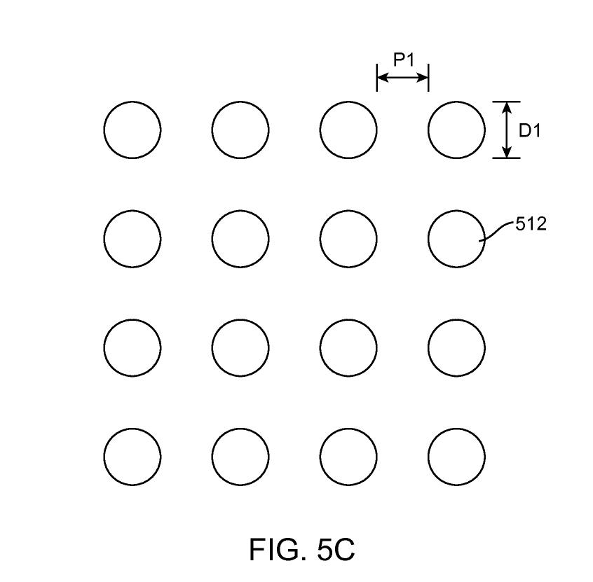

In some variations, the sample may be collected and tested using an array of wells or chambers. A top view of an example of a test well array (500) is shown in FIG. 5A. Each well (510) may be able to accumulate a volume of sample of about 1 mL to about 10 mL, such as 5 mL. For example, each well (510) may be able to accumulate a single sweat bolus for testing. Test well array (500) may be an n1 by m1 matrix of wells, where n1 may be, for example, about 200 to about 500 wells, and m1 may be, for example, about 200 to about 500 wells, and in some variations, n1 is equal to m1 for a square array. The length L8 of test well array (500) may be about 0.5 cm to about 1.5 cm (e.g., 1.0 cm), and the width W3 may be, for example, about 0.5 cm to about 1.5 cm (e.g., 1.0 cm). Referring to FIG. 5B (top view), each well (510) may have a depth of about 20 microns to about 30 microns, a length L10 of about 400 microns to about 500 microns, and/or a width W5 of about 400 microns to about 500 microns. Of course, these are exemplary dimensions, and other suitable dimensions may also be used.

Referring again to FIG. 5B, each well (510) may have an array of posts (512), where the array of posts (512) may occupy about 25% of the well volume. The array of posts (512) may be an n2 by m2 matrix of posts, where n2 is about 5 to about 20, and m2 is about 5 to about 20. The array of posts may also have a length L9 of, for example, about 50 microns to about 150 microns (e.g., 100 microns) and a width W4 of, for example, about 50 microns to about 150 microns (e.g., 100 microns). As shown in FIG. 5C, each post (512) may have a diameter D1 of, for example, about 15 microns to about 35 microns (e.g., 25 microns), and may be spaced PI apart, where PI is, for example, about 15 microns to about 35 microns (e.g., 25 microns). Each post (512) may have a height of, for example, about 40 microns. Once again, it should be understood that all of these dimensions are only exemplary, and other appropriate dimensions may be used.

There may be any number of posts (512) arranged in an array; for example, there may be 4, 9, 16, 25, 49, 64, or 100 posts. FIG. 5C is a top view of posts (512), and shows that the posts are generally circular in cross-section, however, posts (512) may have any suitable shape, such as a rectangular, or triangular cross-sectional shape, or the like. Posts (512) may be solid, or may comprise a lumen in at least a portion of the post. The interior of wells (510) and posts (512) may be coated (e.g., by cross-linking) with a detection reagent, such as a primary binding agent and/or enzyme binding agent, such as reagents commonly used in an enzyme-linked immunoabsorbent assay (ELISA). For example, the interior of the wells and/or the surfaces of the posts may be bound to chemicals that are capable of reacting with the glucose in sweat. In some variations, the top of each post (512) may be coated with a glucose detection reagent to ensure that the reagent is fully exposed to the applied sample.

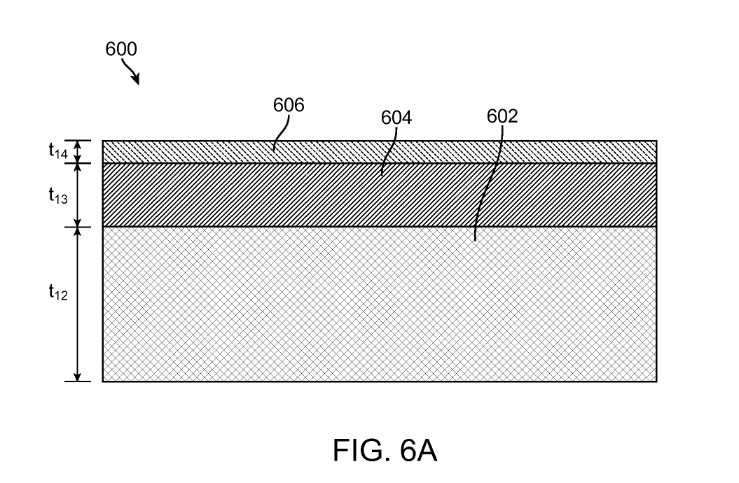

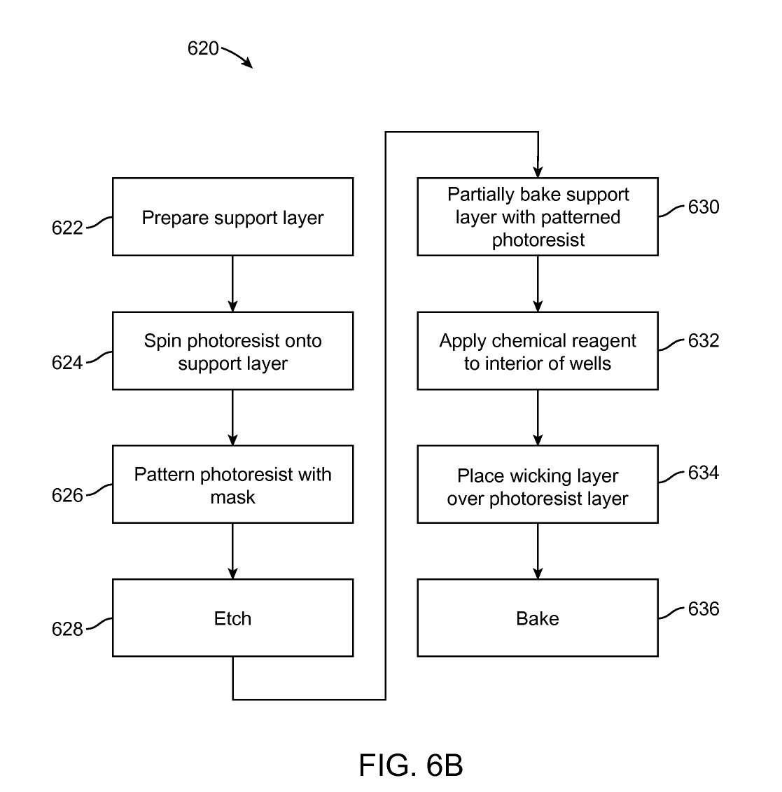

Optionally, test well array (500) may also comprise a hydrophilic porous membrane to wick secreted sweat into well (510). FIG. 6A depicts a cross-sectional view of a portion of a well wall (600) taken at section 6A-6A in FIG. 5B. As shown there, well wall (600) comprises a wicking layer (606), a photoresist layer (604), and a support layer (602). Support layer (602) may be a microporous hydrophobic substrate which passes air but not liquid, for example. The pores in support layer (602) may be about 10 microns to about 40 microns in size (e.g., 20 microns). As shown in FIG. 6A, support layer (602) has a thickness t₁₂. In some variations, t₁₂ may be about 150 microns to about 300 microns. Photoresist layer (604) may be any suitable material, such as SU-8, EPON SU-8, Lithographic Galvanoformung Abformung (LIGA), poly-methyl methacrylate (PMMA), polymethylglutarimide (PMGI), other photoresistive epoxy resins, and any positive or negative photoresistive material that can be etched to form structures with an aspect ratio of about 20 or more. The photoresist layer has a thickness t₁₃, where t₁₃ may be, for example, about 20 microns to about 40 microns. Wicking layer (606) may be a microporous hydrophilic membrane, such as Nuclepore™, and may be placed over photoresist layer (604) to wick secreted sweat into the chambers/wells and to react with the chemistry bound to the interior surfaces of the chamber/wells. Membrane materials are also described, for example, in U.S. patent application Ser. Nos. 11/159,587 (published as US 2006/0004271 A1) and 11/451,738 (published as US 2007/0027383 A1), both of which were previously incorporated herein by reference in their entirety. Wicking layer (606) has a thickness t₁₄, where t₁₄ may be, for example, about 5 microns to about 50 microns.

A testing device including the above-described structures and features may enable the measurement of glucose from the secretion of a single sweat gland anywhere on the skin. As a result, the testing device may allow for completion of a sweat glucose test within a few seconds. In one variation of the above described well array, a sweat bolus may be secreted onto the hydrophilic wicking layer, where the pores draw the sweat bolus into one of the chambers/wells. The sweat bolus may then react with the chemistry that was previously adsorbed into the chamber. In some variations, the chemistry may be any enzyme for glucose detection, and may be capable of changing color to indicate the quantity of glucose in the sample. In certain variations, the chemistry applied in the interior of the chamber may be a reagent used in an ELISA. Once the ELISA is completed in the chamber, an optical system may view each chamber in the array of chambers, and may detect any color changes in each of the chambers. The collected optical data may then be used to determine the quantity of glucose in the sweat bolus by downstream processing (e.g., using an external or embedded computing device), which may be recorded and/or reported to the subject.

### D. Method of Making Test Well Array

Test well array (500) may be made using any suitable technique, for example, using photolithography methods, such as the method (620) shown in FIG. 6B. Method (620) is one possible photolithography method that may be used to form test well array (500), and other photolithography methods, using different photoresists (e.g., EPON SU-8 epoxy resin, LIGA, PMMA, etc.) with different etch techniques (e.g., different chemicals, for varying quantities of time) may be used as appropriate. As shown in FIG. 6B, method (620) comprises preparing a support layer for application of a photoresist (622). The support layer may be any rigid, hydrophilic, microporous material, as described previously. The surface of the support layer may be treated to promote adhesion of photoresist. Next, SU-8 photoresist may be spun onto the support layer to a thickness t₁₂, as described above (624). Then, the photoresist may be patterned with a mask in order to obtain the structures depicted in FIGS. 5A-5C (626). After light in the UV range has been applied to the photoresist, the photoresist may be etched, for example using H₂SO₄ or any other appropriate chemical reagent (628). The etch time may vary depending on the desired depth of the well and height of posts (e.g., FIG. 5B). The patterned photoresist and support layer may then be partially baked (630). The detection reagent (e.g., enzyme/chemical linked with an optically detectable molecule or any ELISA reagent for glucose) may be adsorbed into the interior of the patterned chambers (632). A wicking layer, such as Nuclepore™, may be applied over the photoresist (634), and all layers may be baked (636). In some variations, the detection reagent may be applied after the final bake (636), especially if reagent reactivity may be affected by the final bake. After the final bake (636), any detection reagent that may be on the wicking layer may be removed. Alternatively, the patterned photoresist and support structure may be completely baked after etching (628). After the complete bake, the detection reagent may be applied to the interior of the chambers and dried. The wicking layer may then be applied to the patterned photoresist by electrostatic attraction and/or a vapor adhesive applied to the bottom surface of the wicking layer. The application of the detection reagent to the interior of the chambers may take place before, after, or in addition to any of the steps of method (620), as suitable for preserving the reactivity of the detection reagent.

In other variations, an array of chambers may be formed by crushing or micro-embossing crushed and uncrushed regions into a colorimetric membrane that is reactive to glucose in a sweat bolus. Other appropriate methods may also be used.

### E. Measurement Devices

In a method that includes collecting sweat for glucose concentration analysis, once the glucose in the collected sweat has reacted with the reagent or reagents in the colorimetric membrane, any of a variety of different devices and methods may be used to measure the resulting color. In some variations, an optical system may be used to read the color of the membrane, and to correlate the reading to blood glucose concentration. The optical system may, for example, be relatively precise, easy to use, and/or inexpensive. The particular optical system that is employed may depend, for example, on the dye or dyes that are used, and/or on the pattern of color development in the membrane. In some variations, the optical system will measure one or more optical properties of the test strip, such as reflective, transmissive, absorptive, or emission properties of the membrane of the test strip. Each of these properties may require specific forms of optical illumination and detectors.

In certain variations, the optical system may comprise a light-tight chamber that is configured to retain the test strip. In some variations, the test strip may be manually placed in the chamber. In other variations, a test strip-dispensing mechanism may be integral with the optical system, thereby eliminating the need for any manual intervention. Within the light-tight chamber, the test strip may be positioned (e.g., manually, mechanically, or electrically) so that the region of interest (e.g., a test region containing the sample) is accessible for optical probing.

Optical data obtained from the test strip may be used in a number of ways. For example, optical data may be used to determine whether a sufficient quantity of fluid (e.g., sweat) is present for accurate testing, and/or may be used to analyze the quantity and/or concentration of a target analyte.

Reflectance and transmission readings at single or multiple wavelengths in both the visible and non-visible ranges may be employed. In some variations, fluorescent indicators may be used. In certain variations, relatively simple reflectance measurements may be made using any of a variety of light sources, such as single or multiple light-emitting diodes (LEDs), lasers, and/or laser diodes. Illumination may be at a specific wavelength or wavelengths, or may incorporate a broad range of wavelengths (e.g., depending on the indicator dye that is used in the colorimetric membrane). For example, certain light-emitting indicators (e.g., fluorescent indicators) may emit a stronger light signal if excited by light within a particular range of wavelengths. Some variations of optical systems may illuminate using monochromatic light, or may incorporate a filter that selects for the range(s) of wavelength light (e.g., bandpass, low pass, or high pass filters). The characteristics of the light that is used to illuminate the test strip (e.g., wavelength, intensity, exposure time) preferably are such that the dye provides reliable emissions, but does not bleach the dye indicator.

The light emitted or reflected by a dye indicator may be detected by one or more sensors configured to capture light of the emission or reflected wavelength. For example, the light emitted and/or reflected by the indicator may be detected by one or multiple photodiodes, where the photodiodes may be tuned to detect a narrow or broad band of wavelengths. Reflectance data (e.g., color data) may be obtained by at least one photodiode, as appropriate. In some variations, a wide spectrum light may be used to illuminate the membrane, and light emitted or reflected from the dye indicator may be detected by a charge-coupled device (CCD) or CMOS-based detector. For example, the emitted or reflected light may be detected by a CMOS-based camera or any digital camera which images the membrane on a pixel-by-pixel basis. Alternatively or additionally, the light may be captured on a photographic medium, such as light-sensitive film, or using a reflection densitometer. The image may be monochromatic, or may incorporate light of a range of wavelengths. In other variations, the light emitted and/or reflected from the colorimetric membrane may be recorded over a period of time, in preprogrammed intervals (e.g., using a video camera). The color of the test strip can be measured while the colorimetric membrane is reacting with the sample and changing color (on-meter dosing), or after the colorimetric membrane has completed the color change (off-meter dosing). Time-lapsed image recording may provide additional data that may be used to evaluate the fluid sample, for example, to estimate the sweat rate by monitoring the appearance of colored spots, and may be used to signal whether sufficient sample has been collected (e.g., to signal insufficient or excessive sample volume). Monitoring the appearance of the colored spots (e.g., timing and location) may be used as criteria to distinguish between sweat-derived glucose, and glucose from other sources that do not change rapidly with time.

The detector or detectors may acquire an image of a substantial portion of the test region, or may acquire an image of a small portion of the test region (e.g., a single pixel). When a focal light source is used to image the test strip, such as a laser or pin hole light source, the light beam may be scanned across the test region to generate a full image, or the test strip may be mechanically scanned through the light beam to generate a full image. The scanning procedure may be pre-programmed and/or automated, or may be manual, and subject to real-time adjustment by the user. The scan speed may be selected to achieve a certain resolution suitable for adequately precise analyte detection, and may be adjusted to reduce photo-bleaching and to acquire the image before substantial dye indicator migration. The image data acquired by the detector or detectors may be transmitted and/or stored for processing and analysis, or may be processed in real-time, as described below.

Various optical components may be included to focus light onto the test strip and/or detectors. For example, one or more lenses, mirrors, and/or filters may be employed to direct the path of illuminating and/or emitted light. The optical system and its constituent components may be configured for the illumination and detection of sub-millimeter features. For example, the optical system may be tuned to examine the concentration of an analyte (e.g., glucose) in a sample volume of less than one microliter, where the colored indicator may be on the order of tens or hundreds of microns. Focal light sources, such as lasers, may be suitable for the detection and measurement of sub-millimeter and sub-micron test strip features. The light source, optical components, and detectors may be calibrated as needed to ensure consistently precise measurements for both microliter and nanoliter sample sizes. In some optical systems, calibration may take place at programmed time intervals, or may be initiated by the user.

In certain variations of optical systems, the optical transmission property of the test region may be evaluated. For instance, the optical density of a test region may be measured using a variety of instruments, such as transmission densitometers, infrared transducers and receivers, where some instruments use a scanning optical arrangement and/or others use a fixed optical arrangement. In some optical systems, light emitted from each region of the test strip may be detected by a different detector, and the data may be combined in post-processing and analysis to form a complete image. To this end, the membrane may be scanned, in much the same way as electrophoresis gels are scanned, with the optical density of the colored portions analyzed and the transmission property correlated to glucose concentrations. The optical transmission data from the instrument may be transmitted and/or stored for processing and analysis, as described below.

Optical data collected from a test strip may be stored in a memory buffer, or in an external memory resource (e.g., flash drive, CD/DVD, magnetic tape, etc.) for post-processing. In some cases, the data may contain multiple wavelength lengths (e.g., dichromatic or trichromatic), or may be monochromatic. Monochromatic data may be analyzed for intensity, where the intensity may be denoted as an eight bit value (0 to 255, where 0 is absolute darkness and 255 is maximum brightness). Individual wavelengths of light may be extracted from wide spectrum light, and the intensity of each channel (e.g., red, green, and blue) may be analyzed similarly.

The optical data collected from a test strip may be mapped against a standardized curve or plot that correlates that optical property with the concentration of the analyte. Alternatively or additionally, the optical data collected may be compared with a calibration curve that is obtained prior to analyzing the test sample. For example, the glucose concentration in a sweat sample may be determined based on the optical density of a single wavelength channel extracted from a composite image. In some variations, the glucose concentration may be directly related to the image data. For example, the intensity value per pixel may be correlated to the analyte concentration in the fluid sample. As an example, the intensity value of a given pixel may be proportional to the concentration of glucose in a sweat sample. Alternatively, the intensity value of a given pixel may be proportional to the quantity of the glucose in a sweat sample. Experiments and examples of optical detection techniques used to detect the concentration of glucose in sweat are provided and described below.

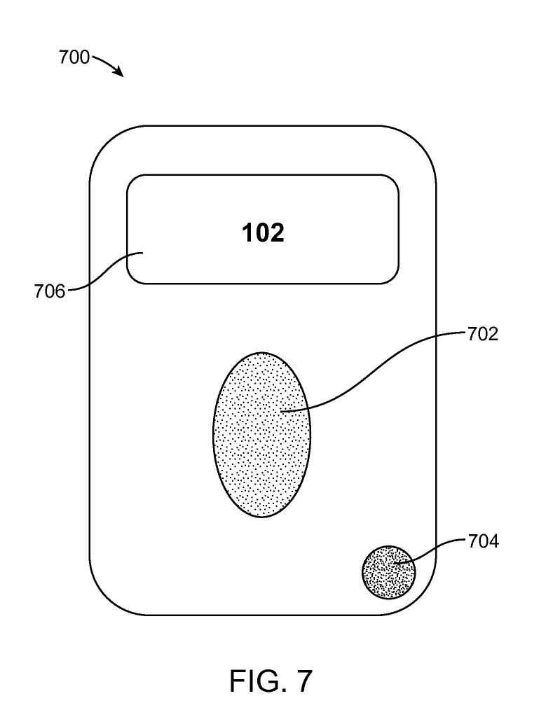

FIG. 7 illustrates a meter (700) that may be used to measure the concentration of glucose in a sample of sweat collected by a test strip. As shown in FIG. 7, meter (700) comprises an optical window (702), a power switch (704), and a display (706). The colorimetric membrane of a test strip containing a fluid sample therein may be placed on top of optical window (702), such that the colorimetric membrane is sufficiently presented to the optical system embedded in meter (700). To ensure adequate contact between the test strip and optical window (702), the user may place a fingertip on top of the test strip to press it into the optical window, and to transfer sample to the colorimetric membrane in the test strip. In some variations, meter (700) may comprise a pressure sensor that informs the user whether sufficient pressure has been applied to obtain an adequate quantity of sweat. After a period of time (e.g., about 20 seconds) has passed, meter (700) may detect spot formation on the colorimetric membrane, and may notify the user (e.g., via a visible or audible signal, such as an audible beep) that his or her finger may be removed from the membrane. The meter may measure the color of the colored region or regions (e.g., spots) on the colorimetric membrane either while the finger is in contact with the membrane, or when the finger is no longer in contact with the membrane, and may thereby determine the glucose concentration in the sweat that caused the colored region or regions to form. The meter may then use a built-in algorithm to correlate the sweat glucose concentration to blood glucose concentration, and may report the resulting blood glucose concentration value to the user. The user may then remove and dispose of the test strip.

Alternatively, sweat may be applied to the test strip before the test strip is inserted into the meter. In this variation, spot formation on the colorimetric membrane may be measured after the user's finger has been removed from the membrane. Of course, while the concentration of glucose in a sweat sample is discussed here, it should be understood that any of the devices, methods, and/or kits described here may be used to detect other analytes, and/or may be used to evaluate other types of fluid samples, as appropriate.

Some variations of a meter may also comprise an embedded optical system, configured to interface with a test strip inserted into the meter. In certain variations, the interface between the embedded optical system may include components that provide illumination of the test strip, and detect light emitted from the test strip. Examples of such components have been described above.

During use, a colorimetric test strip may be optically interrogated to determine the quantity (e.g., volume, concentration) of glucose in the sweat sample. This value may then be presented to the user on display (706). Display (706) may also prompt the user to take specific actions based on the glucose concentration in the sweat sample. For example, the user may be prompted to eat certain foods to increase blood glucose, or to take insulin to reduce blood glucose. After the glucose reading is completed, the test strip may be removed from the meter and disposed.

In some variations, an access port may be used, either as an alternative to, or in addition to, an optical window. The access port may allow for substantial contact of a fingertip to a colorimetric membrane contained in the meter. In such variations, the colorimetric membrane may be in the form of a spool that is turned as each test is conducted, where one spool accumulates used colorimetric membrane material, while another spool retains new colorimetric membrane materials. The access port would allow for unobstructed contact between a skin surface and the reactive layer.

As discussed above, in some variations, a meter or measurement device may include one or more algorithms to convert a sweat glucose concentration value to a blood glucose concentration value. For example, the meter or measurement device may comprise computer-executable code containing a calibration algorithm, which may be used to relate measured values of detected glucose to blood glucose values. In some variations, the algorithm may be a multi-point algorithm, which is typically valid for about 30 days or longer. The algorithm may necessitate multiple capillary blood glucose measurements (e.g., blood sticks) with simultaneous test strip measurements over about a one-hour to about a three-day period. This could be accomplished using a separate dedicated blood glucose meter provided with a glucose measurement device described herein, which comprises a wireless (or other suitable) link to the glucose measurement device. In this way, an automated data transfer procedure may be established, and user errors in data input may be minimized.

Once a statistically significant number of paired data points has been acquired having a sufficient range of values (e.g., covering changes in blood glucose of about 100 mg/dL), a calibration curve may be generated to relate the measured sweat glucose to blood glucose. Subjects (e.g., patients) may perform periodic calibrations checks with single blood glucose measurements, or total recalibrations as desirable or necessary.

Certain variations of glucose measurement devices may also comprise a memory for saving readings and the like. Additionally, glucose measurement devices may comprise a processor configured to access the memory and execute computer-executable code stored therein. It should be understood that glucose measurement devices may include other hardware such as an application specific integrated circuit (ASIC). In addition, glucose measurement devices may include a link (wireless, cable, or the like) to a computer. In this way, stored data may be transferred from a glucose measurement device to a computer for later analysis, etc. Alternatively or additionally, glucose measurement devices may include an interface that is compatible with a mobile device, such as a Blackberry™ or iPhone™ or iPod™ mobile device, where sweat glucose measurements may be recorded and optionally uploaded to a website or remote server in real-time. The sweat glucose data may be analyzed to determine trends in a subject's glucose levels, as well as develop predictive models to aid in glucose management. Trends and models of glucose levels as a function of any variable (i.e., time, disease progression, behavior, caloric intake, etc.) may be displayed on the website that is accessible to a medical professional monitoring the health of the subject and the subject. Glucose measurement devices may also comprise various buttons to control the various functions of the devices and to power the devices on and off when necessary.

### Methods of Measuring Analyte Concentration

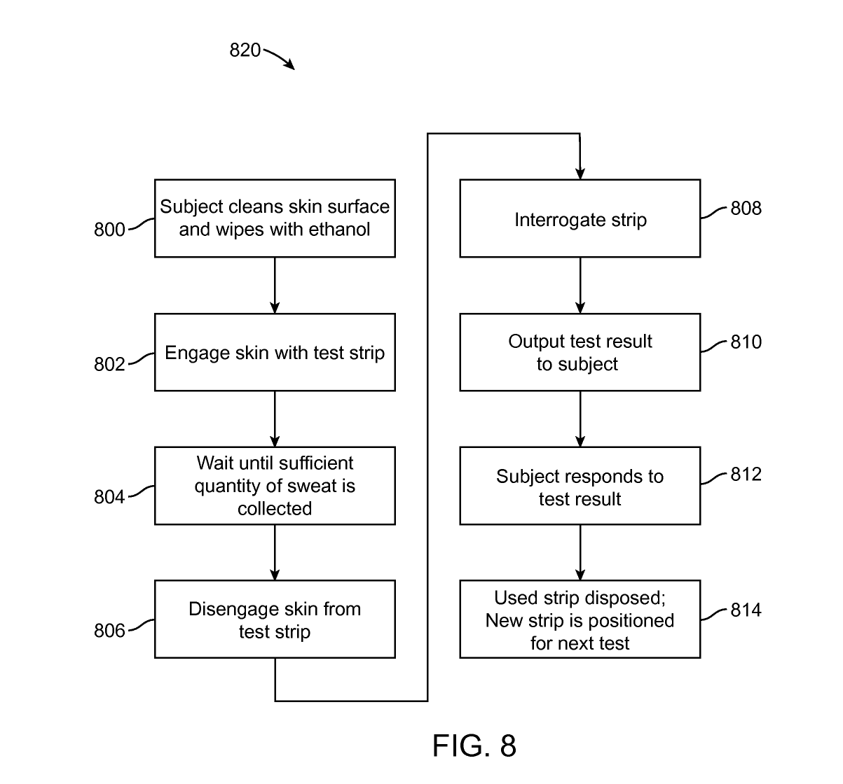

As discussed above, test strips and related devices described here may be used to measure the concentration of glucose in sweat. A test strip comprising a porous membrane such as one of those described above may be used, for example, to collect sweat from the skin surface of a diabetic patient. The test strip may then be evaluated to estimate the blood glucose level of the diabetic patient using the collected sweat. During use, as the sweat enters the pores, one or more analytes in the sweat may react with one or more reagents in the membrane, thereby causing a color to form in the membrane. The color in the membrane may be measured and correlated to glucose concentration in the sweat. The sweat glucose concentration may then be correlated to glucose concentration in whole blood. Hence, methods described here may be used as a substitute for traditional blood glucose monitoring, where samples of blood are obtained by way of a fingerstick. One variation of a non-invasive method (820) is depicted in FIG. 8.

As shown in FIG. 8, first the subject optionally may clean an area of skin to remove residual glucose present at the skin surface (800). Exemplary wipes that may be used are described, for example, in U.S. patent application Ser. No. 10/358,880 (published as US 2003/0176775 A1), the disclosure of which is hereby incorporated by reference in its entirety. For example, the subject may use one or more wipes impregnated with a cleanser that does not interfere with glucose detection and/or a surfactant (e.g., sodium lauryl sulfate (SLS)) that modifies one or more properties of the sweat and/or the skin surface. In some variations, the wipes may contain one or more chemical markers that are identifiable (e.g., using a measurement device) to confirm that the skin was wiped before the sweat was collected by the test strip. Alternatively or additionally, the subject may wipe the skin surface with ethanol to remove unwanted substances from the skin surface. Other sterilization techniques may also be employed to remove substances that may cause an erroneous reading by the test strip or meter.

Next, the subject may hold the test strip against a skin surface (802). While it may not be necessary to do so, in some variations, the subject may attach the test strip to the skin surface. The test strip may be attached to a skin surface in any of a number of different ways. In some variations, the subject may remove a release liner from a bottom surface of the test strip to expose a pressure-sensitive adhesive that may adhere to the skin. Alternatively or additionally, other adhesives (e.g., heat-sensitive or soluble adhesives) may be used. In certain variations, the test strip may be positioned using an elastic band configured to hold the test strip in place. In some variations, the subject may tape the test strip to a skin surface (e.g., using medical tape), and/or may hold the test strip to a skin surface. In certain variations, the test strip may be held in place on the skin using a “watch-like” device. In other variations, the test strip may be retained within the meter, where the meter comprises an access port. The subject may contact a portion of skin (e.g., a finger tip) to the test strip by placing the finger through the access port and pressing against the test strip. Alternatively, the membrane portion of the test strip may protrude from the meter to ensure sufficient contact with a subject's skin.

The subject's skin surface may be engaged with the test strip for a period of time so that a sufficient quantity of sweat is collected (804). The meter may employ optical means (such as those described previously) to determine the volume of sweat collected. A program or algorithm may determine whether the collected volume is sufficient, and indicate an instruction to the subject to maintain contact with the test strip, or disengage from the test strip. In some variations, skin may be engaged with a test strip for a pre-determined amount of time that has been shown to be sufficiently long to collect a testable quantity of sweat. For example, the subject may contact his/her skin to the test strip for a period of about 2 seconds to about 30 seconds. In some variations, the subject may contact his/her skin to the test strip for about 1 minute to about 30 minutes. Alternatively or additionally, a colorimetric test strip may comprise a dye indicator that changes its optical qualities (e.g., changes color and/or opacity) to signal that a sufficient quantity of sweat sample has been collected. The optical change may be detected by an optical system, or by visual inspection.

Once the test strip has collected a sufficient volume of sweat, the subject may disengage from the test strip (806) and use a measurement device (e.g., a meter) to interrogate the test strip and quantitatively measure the sweat glucose concentration (808). In some variations, the test strip may be removed from the skin and inserted into, or otherwise contacted with, the measurement device (for example, as shown in FIG. 7). In other variations, the measurement device measures the sweat glucose concentration (808) while the test strip is in contact with the subject's skin. Alternatively or additionally, the glucose measurement device may be placed in contact with the test strip (for example, via an optical port as shown in FIG. 7). In variations in which the test strip is retained in the meter, the meter may directly interrogate the membrane of the test strip by measuring chemical, electrical, or optical signals. For colorimetric test strips, an optical system as described previously may be used.

During interrogation (808), the sweat glucose concentration may be obtained, and if so desired, may then be used to derive a blood glucose concentration. The concentration of other analytes may also be determined, as enabled by the colorimetric membrane of the test strip. The concentration of the target analyte(s) may be output to the patient (810) using, for example, a display and/or sound speaker. Optionally, the measurement device may also issue instructions to the subject based on the concentration of the target analyte(s), where the instructions are pre-programmed by a physician or healthcare professional. The subject may respond to the test result (812). For example, based on the sweat glucose concentration and/or blood glucose concentration, the subject may be instructed to self-administer insulin. Once the testing is completed, the subject may remove the test strip from the measurement device and dispose of the test strip (814). In some variations where the test strip is retained by the measurement device, the device may then advance the used test strip and present an unused test strip for the next test.

It should be noted that in some variations, method (820) may be performed by someone other than the subject (e.g., a medical/healthcare professional) on the subject's behalf. Additionally, the above description is directed to employing test strips to obtain a sweat glucose concentration from skin surface sweat. It should be understood that method steps may be removed or added, and/or repeated as appropriate.

In some variations, the devices, methods, and kits described here may be configured for use with measuring an analyte in a specific concentration range in a fluid sample. For example, in certain variations in which sweat glucose concentration is being evaluated, the expected concentration range may be from about 0.1 mg/dL to about 10 mg/dL (e.g., about 0.5 mg/dL to about 4 mg/dL). Accordingly, the devices used to measure the sweat glucose concentration may be designed or otherwise configured to measure the concentration in that expected range. In some variations, devices, methods, and/or kits described here may be used to measure the concentration of an analyte in a fluid sample when the expected concentration is up to about 500 mg/dL (e.g., from about 0.1 mg/dL to about 500 mg/dL, from about 0.1 mg/dL to about 400 mg/dL, from about 0.1 mg/dL to about 300 mg/dL, from about 0.1 mg/dL to about 200 mg/dL, from about 0.1 mg/dL to about 100 mg/dL, from about 0.1 mg/dL to about 50 mg/dL, from about 0.1 mg/dL to about 10 mg/dL, from about 0.1 mg/dL to about 4 mg/dL, from about 0.5 mg/dL to about 500 mg/dL, from about 0.5 mg/dL to about 400 mg/dL, from about 0.5 mg/dL to about 300 mg/dL, from about 0.5 mg/dL to about 200 mg/dL, from about 0.5 mg/dL to about 100 mg/dL, from about 0.5 mg/dL to about 50 mg/dL, from about 0.5 mg/dL to about 10 mg/dL, from about 0.5 mg/dL to about 4 mg/dL, from about 50 mg/dL to about 500 mg/dL, from about 50 mg/dL to about 400 mg/dL, from about 50 mg/dL to about 300 mg/dL, from about 50 mg/dL to about 200 mg/dL, from about 50 mg/dL to about 100 mg/dL). The expected concentration range of an analyte will likely depend, for example, on the type of analyte and/or the type of fluid sample involved.

While both detection of an analyte in a fluid sample and measurement of the concentration of the analyte in the fluid sample have been described, some variations of methods may comprise detecting an analyte in a fluid sample without also measuring the concentration of the analyte in the fluid sample. Additionally, while measurement of the concentration of an analyte in a sweat sample and correlation of the sweat concentration measurement to a blood concentration measurement have been described, certain variations of methods may comprise measuring the concentration of an analyte in a first fluid sample (e.g., sweat) without later correlating the measurement to a concentration of the analyte in a second, different fluid sample (e.g., blood). For example, a diabetic may use a sweat glucose concentration measurement to determine whether to administer insulin, and therefore may not need to convert the sweat glucose concentration value to a blood glucose concentration value.

In certain variations, a relatively small sample of sweat may be collected and evaluated. This may be advantageous because, for example, it may result in a short procedure time. Moreover, it may allow relatively small test strips to be used. Such relatively small test strips may, for example, be easily transportable and/or inexpensive to produce.

In some variations, a test strip may be used to determine the concentration of glucose in a sample of sweat having a volume of about 220 picoliters to about 0.01 microliter (e.g., about 1 nanoliter to about 10 nanoliters, or about 0.001 microliter). The volume of sweat collected may be determined in part by the material composition and structure of the portion of the test strip that directly contacts the skin surface (e.g., the spreading layer, and/or the membrane). Some test strip membranes may have a structure and material composition configured to obtain the volume of one, and only one, sweat secretion of a given sweat gland. This may be achieved, for example, using an array of chambers where each chamber is capable of completing a measurement of the glucose in a sweat secretion and of retaining a given volume of a fluid sample (e.g., about 1 nanoliter of a sweat sample). The reactive dye indicator in each chamber may be capable of detecting the quantity of glucose in that given volume of sweat. The concentration of glucose may be determined by dividing the quantity of glucose measured by the volume of sample collected. This computation may be completed for a single chamber, or for multiple chambers in an array.

### EXAMPLES

The following examples are intended to be illustrative and not to be limiting.

### Example 1

### Evaluating Colorimetric Membranes from Test Strips

OneTouch® SureStep® test strips (from LifeScan, Inc.) were purchased from pharmacies and disassembled to obtain their colorimetric membranes. According to their package inserts, the colorimetric membranes included a reagent that reacts with glucose to cause a detectable color change.

Three different types of fluid samples were applied to the test regions of the colorimetric membranes removed from the test strips: (1) aqueous glucose solutions of known concentration, (2) contrived sweat (i.e., a solution of salt and glucose meant to simulate sweat), and (3) sweat from human subjects. The results of the glucose solution tests will be described in Example 1, and the results of the human sweat tests will be described in Example 2 below.

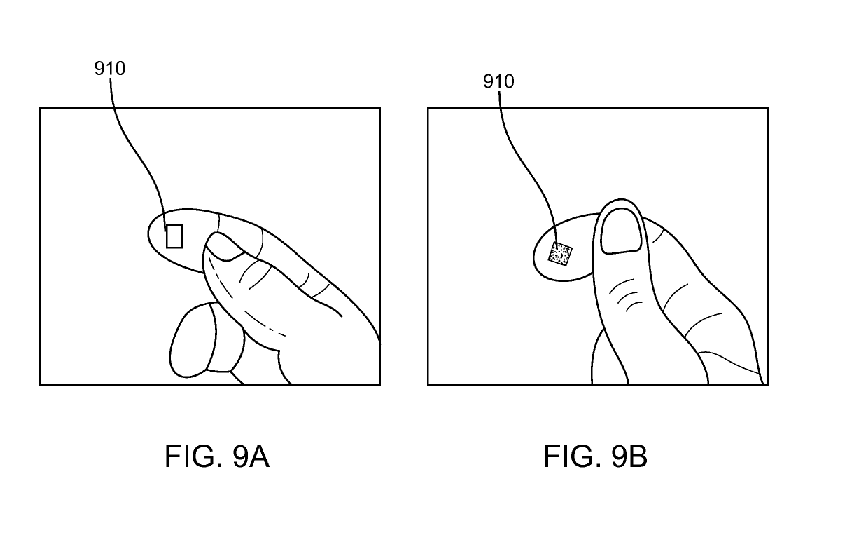

FIG. 9A shows that prior to contacting a fluid sample, colorimetric membrane (910) had certain optical properties (i.e., generally light in color and translucent). A thin film of sucrose solution was then applied to the finger tip and thumb of a subject, and colorimetric membrane (910) was squeezed between the finger tip and thumb for 30 seconds. After 30 seconds, the optical properties of colorimetric membrane (910) changed (i.e., turned blue and more opaque), as shown in FIG. 9B.

This experiment suggests that the glucose in the sucrose solution on the surface of the finger and thumb quickly migrated into the pores of colorimetric membrane (910), and that the colorimetric membrane may be suitable for measuring the glucose concentration of a thin film of liquid sample on the surface of skin.

### Example 2

### Evaluating Glucose Concentration in Sweat Excreted by a Sweat Gland

A colorimetric membrane was obtained from a OneTouch® SureStep® test strip (from LifeScan, Inc.), and its ability to detect glucose in unstimulated sweat was evaluated.

First, a finger tip and thumb of a subject were washed with soap and water, and then wiped with ethanol.

Next, a portion of the colorimetric membrane was squeezed between the finger tip and thumb of a subject. The process was repeated for additional colorimetric membranes from OneTouch® SureStep® test strips, varying the amount of squeezing time. The time in which colorimetric membrane (1000) was squeezed was varied.

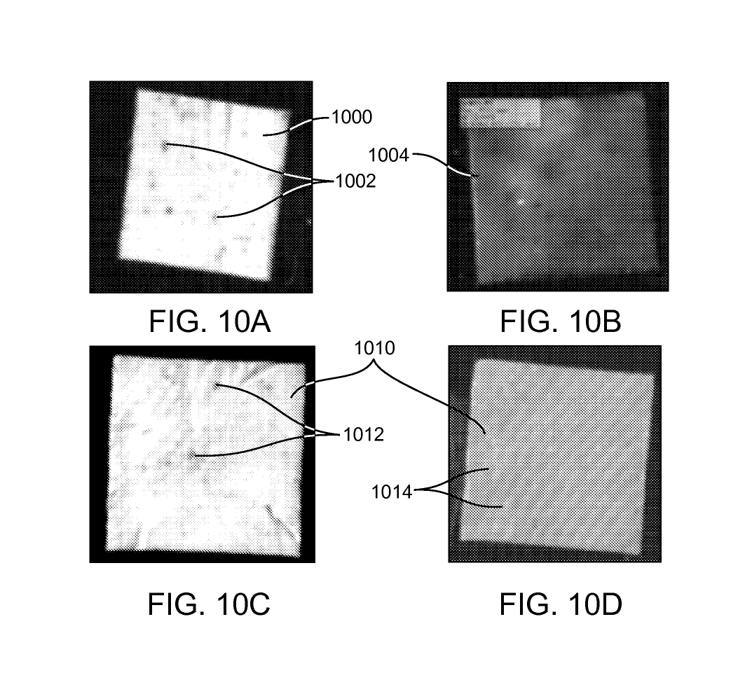

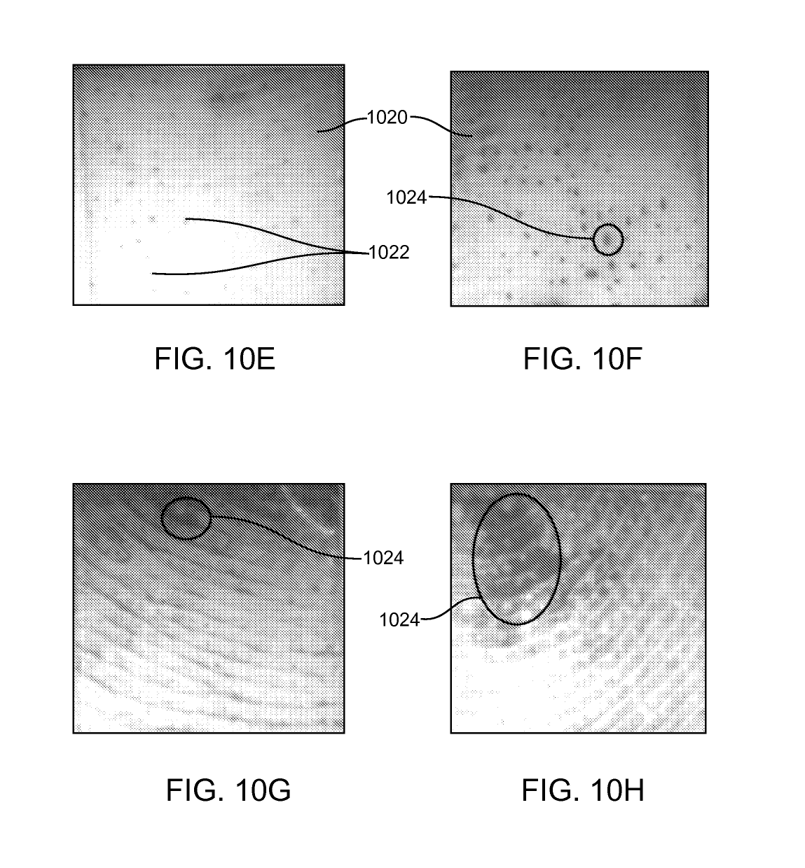

FIG. 10A depicts a colorimetric membrane (1000) that was squeezed between the finger tip and thumb for 5 seconds. After squeezing colorimetric membrane (1000) for 5 seconds, sweat entered the membrane and reacted with the reagent in the colorimetric membrane, forming bright blue spots (1002) corresponding to the locations where sweat glands deposited sweat onto the colorimetric membrane.

FIG. 10B depicts another colorimetric membrane (1004) after being squeezed for 60 seconds. After 60 seconds, sufficient sweat had entered the colorimetric membrane to turn the entire surface blue.

FIGS. 10C and 10D depict an additional colorimetric membrane (1010), where the top side (where the average pore size was about 20 microns) was wrapped with a layer of Parafilm®, leaving only the bottom side (where the average pore size was about 0.2 micron) available for applying a test sample.

Colorimetric membrane (1010) was relatively lightly contacted with a skin surface, with only enough pressure to ensure physical contact.

FIG. 10C shows that after 10 seconds of relatively light contact, blue spots (1012) began to appear, where the blue spots may have corresponded to individual sweat glands. It is believed that the intensity of spots such as these may be analyzed, for example, to determine the glucose concentration in the sweat secreted by a particular sweat gland.

FIG. 10D shows that after 30 minutes of relatively light contact, blue streaks (1014) formed in the shape of a fingerprint. It is believed that such a fingerprint may be used to uniquely identify the test result as belonging to a particular subject (e.g., thereby ensuring that the data collected is authentic).

FIGS. 10E-10H depict a colorimetric membrane (1020) where the top side was sealed with Parafilm®, and the bottom side was contacted with a skin surface. Here, colorimetric membrane (1020) was squeezed between a finger tip and a thumb. The squeeze time was varied (2 seconds, 5 seconds, 60 seconds, and 120 seconds) for each of the panels in FIGS. 10E-10H.

As shown in FIG. 10E, sweat secreted by individual sweat glands could migrate into the membrane and react with the reagent within 2 seconds. In FIG. 10E, each spot (1022) corresponds to an individual sweat gland.

Referring to FIG. 10F, after 5 seconds, more spots appeared, some accompanied by a diffuse distribution (1024) of dye.

By 60 seconds (FIG. 10G) and 120 seconds (FIG. 10H), the diffusive dye effect was more pronounced, and may have represented the migration of indicator dye, or the oversaturation of the colorimetric membrane. In post-processing, the diffuse dye staining may be subtracted out to permit analysis of spots (1022), each of which represents the sweat glucose signal from a sweat gland. Additionally, it is believed that contacting the colorimetric membrane to the skin surface for about 2 seconds to about 10 seconds may prevent excessive indicator dye spreading, as well as contamination from other glucose sources. For example, sweat glucose may be distinguishable from skin surface glucose.

### Example 3

### Evaluating Sensitivity of Colorimetric Membranes

Membranes were removed from OneTouch® SureStep® test strips (from LifeScan, Inc.) as described in Example 1 above, and mounted on a base, so that they could be fed into an inkjet printer.

Inkjets and micropipettes were then used to dispense glucose solutions onto the membranes, and the color of the reacted membranes was measured.

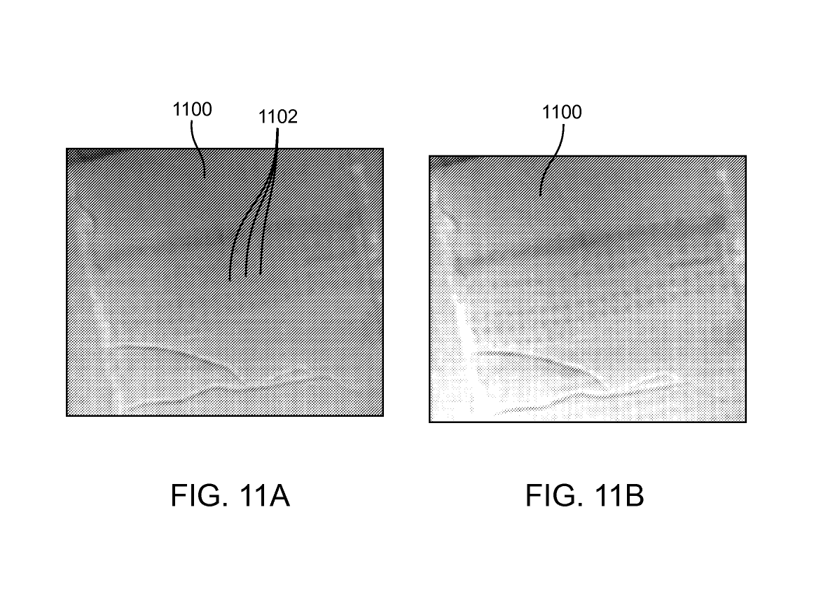

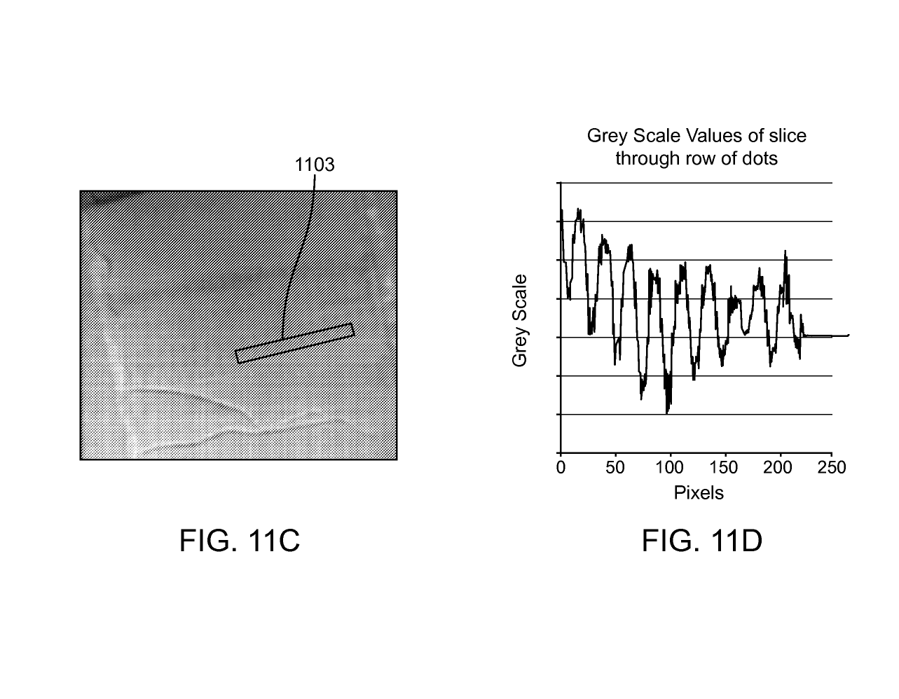

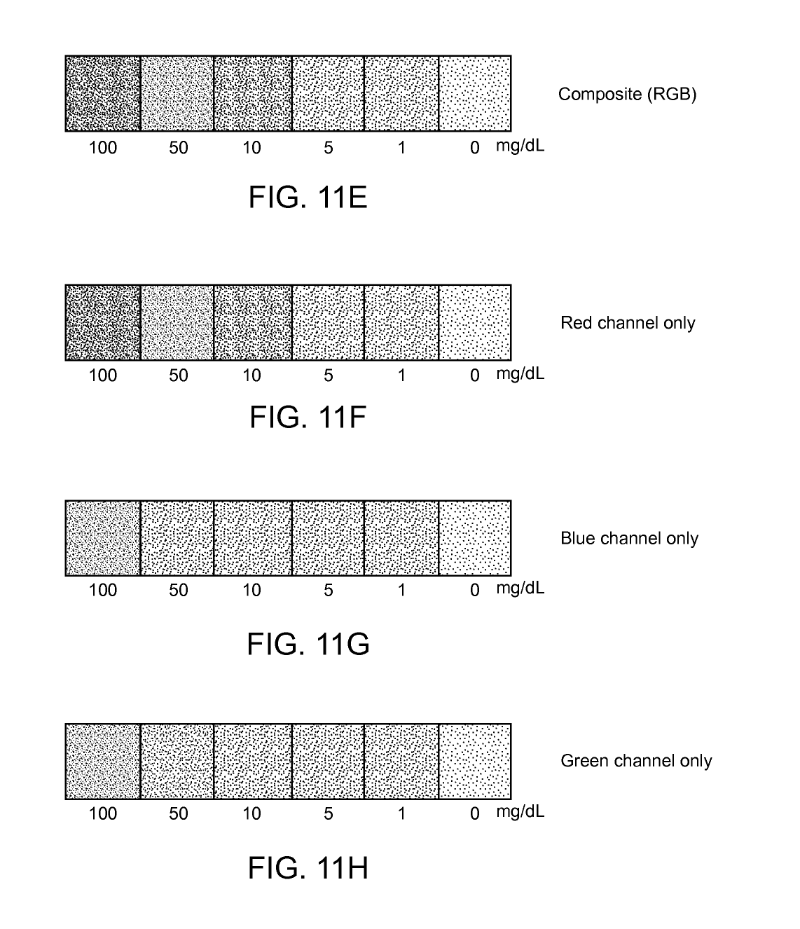

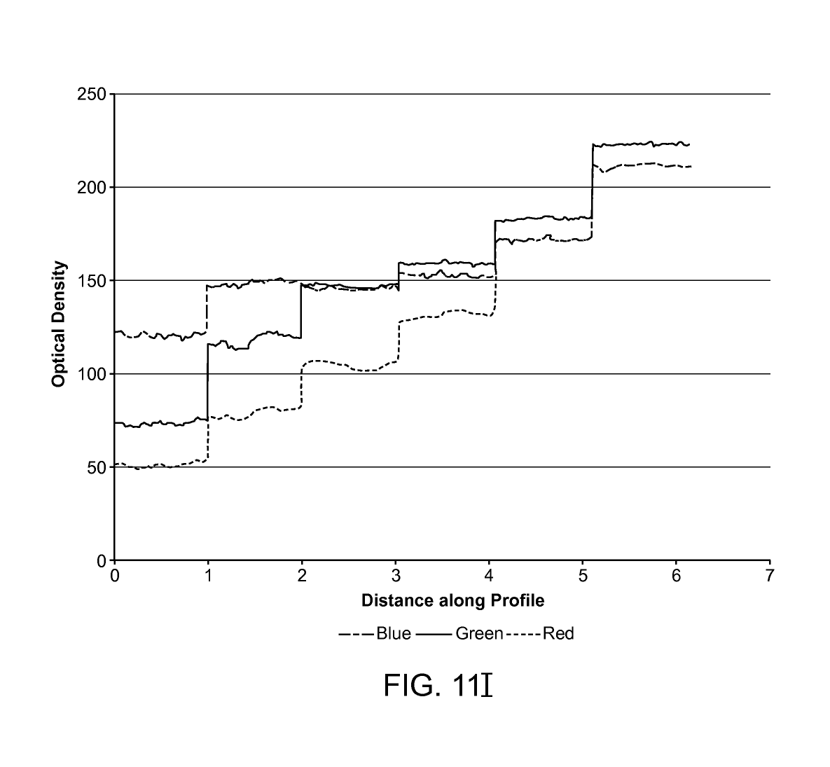

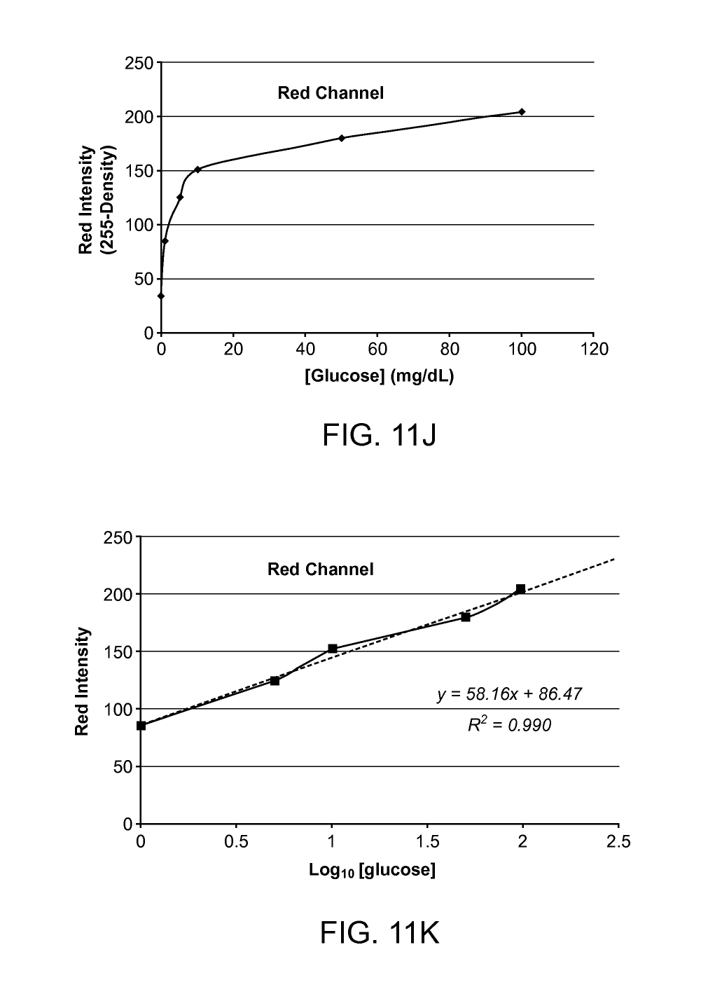

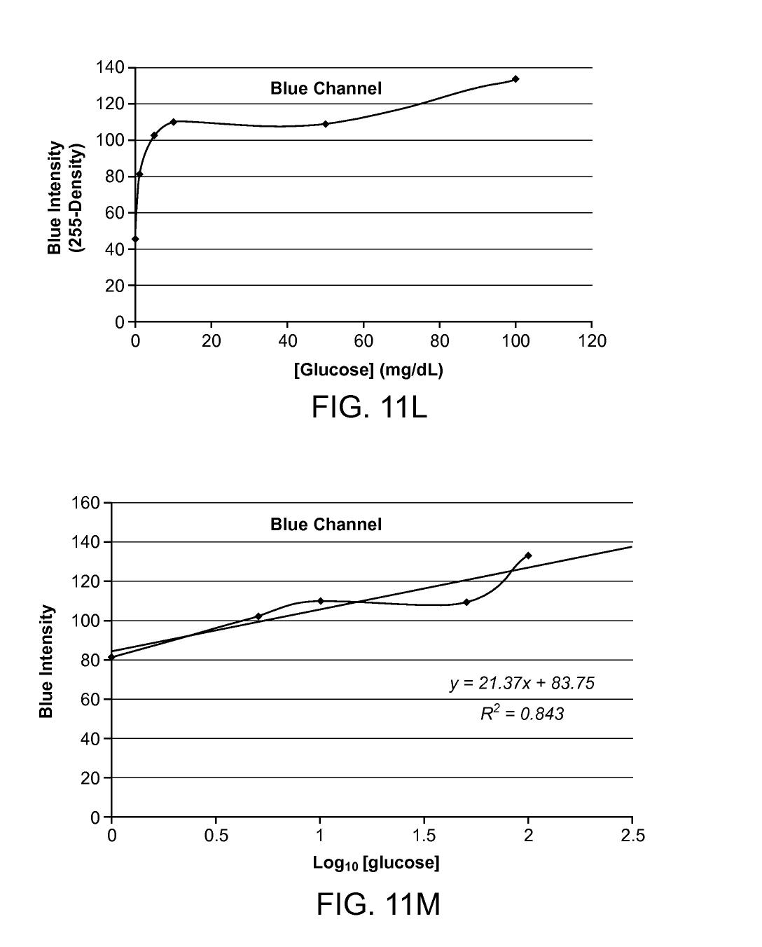

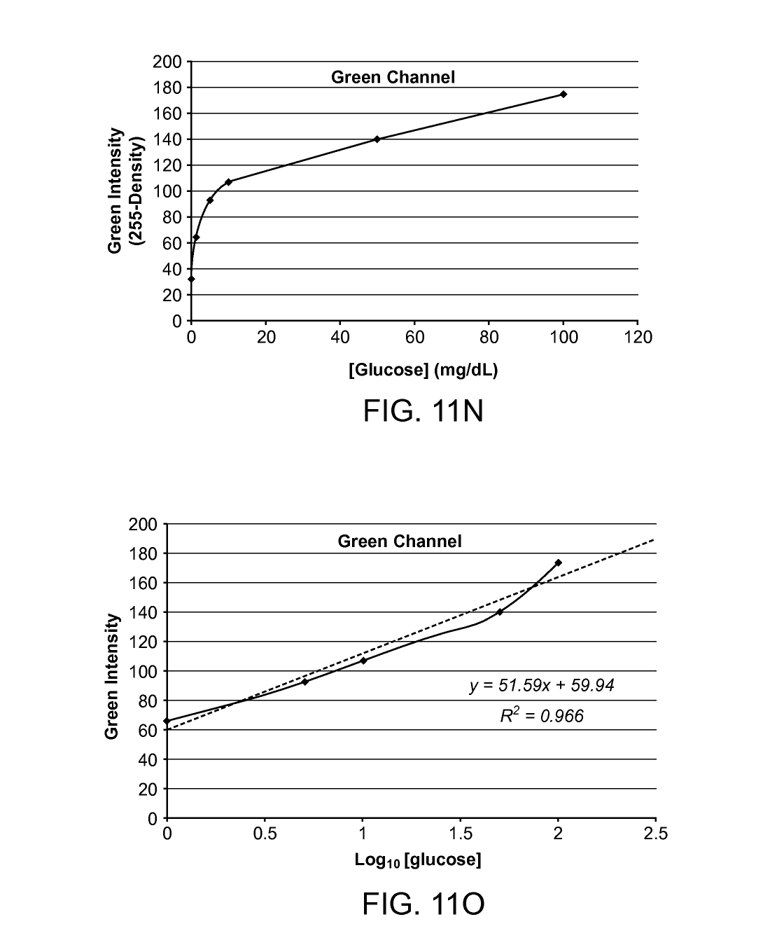

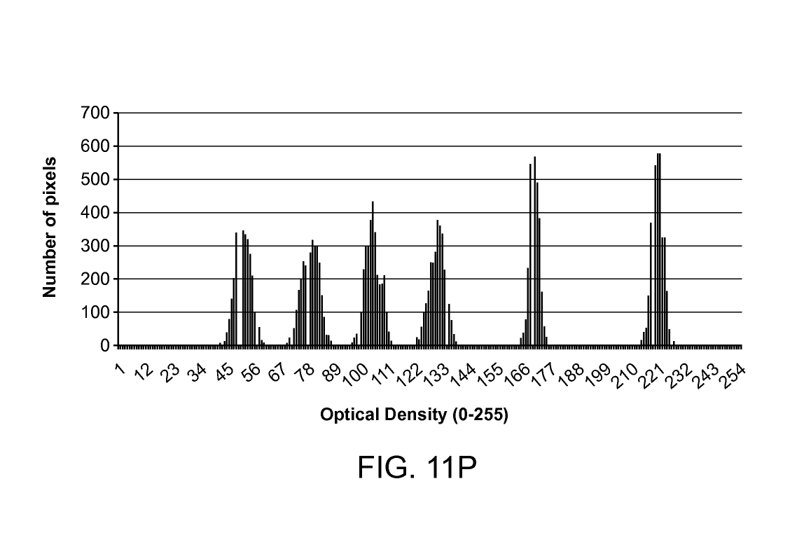

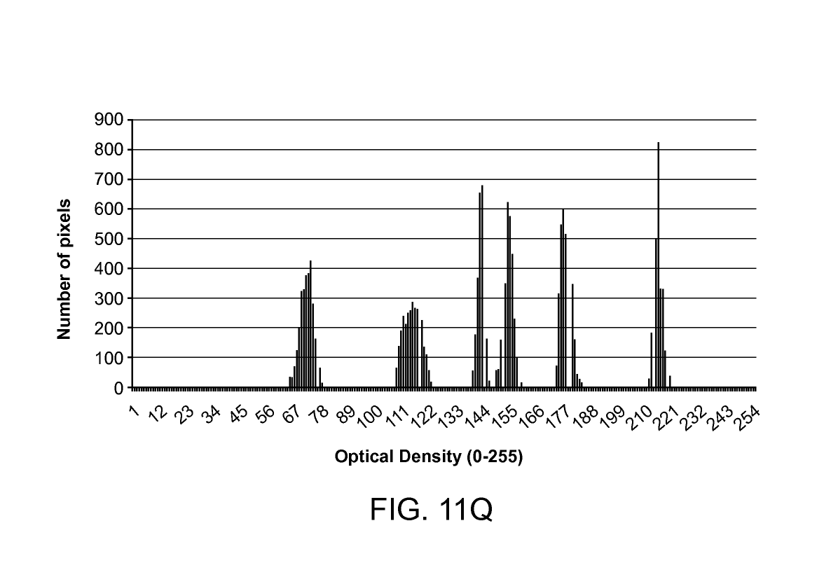

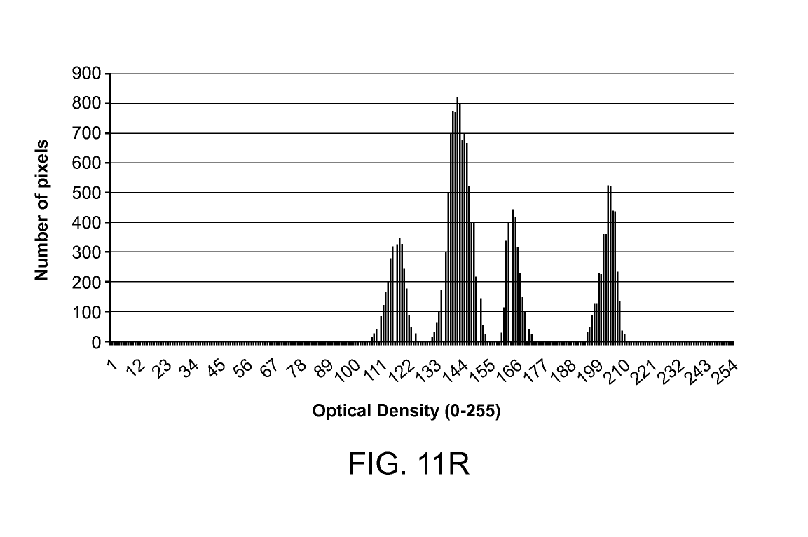

FIGS. 11A-11C depict a colorimetric membrane (1100) from one of the test strips. Small quantities of a solution with a known glucose concentration were applied to colorimetric membrane (1100) with an inkjet printer. More specifically, using a commercially available inkjet head (part number 51612A, from Hewlett-Packard), a 5 mg/dL glucose solution was applied onto the bottom portion (where the pore size is about 0.2 micron) of colorimetric membrane (1100). The approximately 220 picoliter drops were dispensed such that they were approximately 250 microns apart (center to center spacing). The drops had volumes in the same order of magnitude as the drops that pulse out of sweat glands in the epidermal ridges of a finger. As mentioned previously, it is estimated that 1 nanoliter droplets are periodically excreted by sweat glands in the epidermal ridges of the fingers.

As seen in FIG. 11A, which is an RGB (red-green-blue) composite image, spots (1102) formed at the location of glucose solution deposition. FIG. 11B is the red video channel of the frame illustrated in FIG. 11A.

FIG. 11C depicts a group of spots (1103) from the image in FIG. 11B that have been selected and analyzed for grey scale intensity.

FIG. 11D is a plot of the grey scale intensity of the spots selected in FIG. 11C as a function of pixels. FIG. 11D shows that the grey scale intensity of a horizontal slice through the row of spots (1103) from FIG. 11C (where an intensity value of zero is absolute darkness, and an intensity value of 255 is maximum brightness) varies by about plus or minus 2.5%. Grey scale intensity might be a way to measure the intensity of color development in the colorimetric membrane.

FIGS. 11A-11D suggest that a very small volume of 5 mg/dL glucose solution may cause measurable color change in a colorimetric membrane.

### Example 4

### Calibrating Color Changes in a Test Strip to Glucose Concentration

Color changes in a colorimetric membrane may be calibrated to a glucose concentration.

Six colorimetric membranes were obtained from a OneTouch® SureStep® test strip as described in Example 1.

A 5 microliter drop of glucose solution was applied to each test strip, where the glucose concentration was different for each strip (100, 50, 10, 5, 1, or 0 mg/dL of glucose).

After developing the colorimetric membranes for about 2 minutes, a camera module was used to capture an image of the colorimetric membranes. The camera module was IV-CCAM2, with a normal lens, backlight compensation OFF, manual shutter at a speed of 1/60 second, and white balance AWC calibrated against a white background. The colorimetric membranes were illuminated by a light source (Dolan-Jenner MI-150, quartz-halogen, 3200K, color temperature, intensity 80% of max, backlight compensation OFF), using a microscope (Optem). The light source was applied with a dual-arm fiber optic head without focusing lenses, where both fiber optic heads shine into stack of two inverted coffee filters with a hole punched in the center for optics.

The image for each of the six test strips was cropped in the center (100×100 pixel patch).

The six cropped images were analyzed with the ImagJ program (NIH) for optical density (pixel value of zero for total darkness, and 255 for maximum brightness). A profile with the six cropped images (from an image with red, green and blue channels) is shown in FIG. 11E.

The red, green, and blue channels may be extracted and analyzed separately. Thus, FIGS. 11F-11H show the red, green, and blue component (respectively) of the composite profile in FIG. 11E.

The optical density for each component was plotted against glucose concentration, thereby calibrating an optical change in the colorimetric membrane with glucose concentration.

FIG. 11I plots the optical density of a horizontal line drawn through each profile in FIGS. 11F-11H (optical density encoded by 8 bits, where zero is absolute darkness, and 255 is maximum brightness) vs. distance along the profile. As the concentration of glucose in the solution varies across the profile, the optical density of each channel also varies.

The plots from FIGS. 11F-11H were used to derive the plots in FIGS. 11J-11O, which plot the relationship between the optical intensity of a single channel vs. glucose concentration (or base 10 logarithm of glucose concentration).

A linear approximation was obtained for each channel, where the slope of the best-fit line indicates the sensitivity of that channel to glucose concentration. A larger slope indicates that for a given magnitude change in glucose concentration, a greater change in optical density occurs to signal that change. As shown, the red channel has the largest slope, while the blue channel has the smallest slope, which indicates that the red channel signals changes in glucose concentrations with greater sensitivity.

The sensitivity of each channel to glucose concentration is also shown in histograms depicted in FIGS. 11P-11R. To obtain these histograms, the number of pixels of a particular optical density in a profile of a single channel was counted.

FIG. 11P shows the image data for the red channel, where there are clearly six peaks, with each peak corresponding to one of the six test strips to which different solutions with different glucose concentrations were applied.

FIG. 11Q shows the image data for the green channel, where the six peaks are evident, corresponding to each of the six different glucose concentrations. However, the separation between the peaks centers around density values of about 150 and 160, and may be difficult for an optical algorithm to discern.

FIG. 11R shows the image data for the blue channel, where only four peaks are seen, which indicates that the difference between optical densities for different glucose concentrations may not be sufficient here to map optical density to glucose concentration.

### Kits

Also described here are kits. The kits may include one or more packaged test strips, either alone, or in combination with other test strips, one or more glucose measurement devices, and/or instructions. Typically the test strips may be individually packaged in sterile containers or wrappings, and may be configured for a single use. In some variations, multiple test strips may be individually sealed within one sterile container or wrapping. Additionally, some kits may comprise multiple test strips that test for the same analyte, and/or may comprise multiple test strips that test for different analytes.

While the devices, methods, and kits have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the described variations.

1. A method for measuring the concentration of an analyte in sweat of a subject, comprising:
contacting a colorimetric membrane with a skin surface of the subject, wherein at least a portion of the membrane is configured to collect a volume of sweat from the skin surface; and
analyzing the at least a portion of the colorimetric membrane to determine the concentration of the analyte in the collected volume of sweat.

2. The method of claim 1, wherein analyzing the colorimetric membrane to detect the concentration of the analyte in the collected volume of sweat comprises using an optical system to evaluate optical absorption or reflection of the colorimetric membrane.

3. (canceled)

4. (canceled)

5. The method of claim 1, wherein analyzing the colorimetric membrane to detect the concentration of the analyte in the collected volume of sweat comprises using an optical system to evaluate intensity of multispectral or monochromatic light reflected from or transmitted through the colorimetric membrane.

6-16. (canceled)

17. The method of claim 1, wherein the analyte comprises glucose.

18. The method of claim 17, further comprising estimating the concentration of glucose in blood of the subject from the sweat of the subject.

19. The method of claim 17, wherein concentration of glucose in blood of the subject is calculated using at least one algorithm that converts the concentration of glucose in sweat to the concentration of glucose in blood.

20. The method of claim 17, wherein the colorimetric membrane comprises a first component that converts glucose to hydrogen peroxide.

21. (canceled)

22. The method of claim 20, wherein the colorimetric membrane further comprises a second component that detects the hydrogen peroxide.

23. (canceled)

24. (canceled)

25. The method of claim 22, wherein the colorimetric reactive membrane further comprises a third component comprising an indicator that changes color in the presence of hydrogen peroxide.

26. The method of claim 25, wherein the indicator comprises an oxidizable dye or a dye couple.

27-31. (canceled)

32. A system for indicating a concentration of an analyte in a subject's sweat, comprising:
a colorimetric membrane configured to contact with a skin surface of the subject and collect a volume of sweat from the skin surface,
wherein the colorimetric membrane comprises a matrix comprising one or more reagents that to come into contact with the collected volume of sweat such that the regents react with the analyte found in the volume of sweat.

33. The system of claim 32, further comprising at least one spreading layer configured to distribute the volume of sweat on the colorimetric membrane.

34. The system of claim 33, wherein the spreading layer comprises one or more pores configured to allow direct fluid connection through the spreading layer.

35. The system of claim 32, further comprising at least one wicking layer and at least one sink layer, wherein the wicking layer is configured to draw an excess of the volume of sweat to the sink layer.

36. (canceled)

37. (canceled)

38. The system of claim 32, wherein the analyte comprises glucose.

39. The system of claim 38, wherein the colorimetric membrane comprises a first component that converts glucose to hydrogen peroxide.

40. (canceled)

41. The system of claim 39, wherein the colorimetric membrane further comprises a second component that detects the hydrogen peroxide.

42. (canceled)

43. The system of claim 41, wherein the colorimetric membrane further comprises a third component, wherein the reagent in the third component comprises at least one indicator that changes color in the presence of hydrogen peroxide.

44. The system of claim 43, wherein the indicator comprises an oxidizable dye or a dye couple.

45. (canceled)

46. A system for determining concentration of an analyte in sweat of a subject, comprising:
a device configured to determine the concentration of an analyte by evaluating optical characteristics of a colorimetric membrane,
wherein the colorimetric membrane is configured to (a) contact with a skin surface of the subject, and (b) collect a volume of sweat from the skin surface, wherein the concentration of the analyte in sweat can be determined optically.

47-60. (canceled)
